(19) United States
(12) Patent Application Publication
(10) Pub. No.: US 2025/0340826 A1
(43) Pub. Date: Nov. 6, 2025

(54) IMPROVED OLIGOSACCHARIDE PRODUCTION IN YEAST

(71) Applicant: Amyris, Inc., Emeryville, CA (US)

(72) Inventors: Jason BRICE, Emeryville, CA (US); William E. DRAPER, Emeryville, CA (US); Christopher F. MUGLER, Emeryville, CA (US); Nikita JAYANT KUMAR OJHA, Emeryville, CA (US); Dominic PINEL, Emeryville, CA (US); Jessica WALTER, Emeryville, CA (US)

(21) Appl. No.: 18/869,133

(22) PCT Filed: May 11, 2023
(86) PCT No.: PCT/US2023/066892

§ 371 Date: Nov. 25, 2024

Related U.S. Application Data
(60) Provisional application No. 63/345,957, filed on May 26, 2022.

(51) Int. Cl. C12N 1/18; C07K 14/395; C12P 19/00; C12R 1/865

(52) U.S. Cl. CPC C12N 1/18; C07K 14/395; C12P 19/00; C12R 2001/865

(57) ABSTRACT

Provided herein are genetically modified yeast cells capable of producing one or more human milk oligosaccharides (HMOs) and methods of making such cells. The yeast cells are engineered to comprise a heterologous nucleic acid encoding a variant ABC transporter protein YOR1 from Saccharomyces cerevisiae with improved 2′-fucosyllactose export activity and one or more heterologous nucleic acids that encode enzymes of a HMO biosynthetic pathway. Also provided are fermentation compositions including the disclosed genetically modified yeast cells, and related methods of producing and recovering HMOs generated by the yeast cells.

### CROSS-REFERENCE TO RELATED APPLICATION

The present application claims the benefit of U.S. Patent Application Ser. No. 63/345,957, filed May 26, 2022, entitled “Improved Oligosaccharide Production in Yeast,” the disclosure of which is hereby incorporated fully by reference into the present application.

### SEQUENCE LISTING

The instant application contains a Sequence Listing which has been submitted electronically in XML format and is hereby incorporated by reference in its entirety. Said XML copy, created on May 10, 2023, is named “AM14800PCT_Sequence_Listing” and is 36 k kilobytes in size.

### BACKGROUND OF THE INVENTION

Human milk oligosaccharides (HMOs) are the third most abundant component of human milk, with only lactose and lipids present in higher concentrations. More than 200 different species of HMOs have been identified to date in human milk. There is growing evidence attributing various health benefits to these milk compounds. Exemplary benefits include the promotion of the growth of protective intestinal microbes such as bifidobacteria, an increase in protection from gastrointestinal infections, a strengthening of the immune system, and an improvement in cognitive development. Because HMOs are not found in other milk sources, e.g., cow or goat, the only source of HMOs has traditionally been mother's milk. In efforts to improve the nutritional value of infant formula and expand the use of HMOs for child and adult nutrition, there has been an increased interest in the synthetic production of these compounds.

### BRIEF SUMMARY OF SOME ASPECTS OF THE INVENTION

The present disclosure is based, at least in part, on the discovery that particular variants of the adenosine triphosphate (ATP)-binding cassette (ABC) transporter polypeptide YOR1 exhibit the ability to export human milk oligosaccharides (HMOs) across cell membranes more effectively compared to the parental Sc.YOR1 polypeptide. Moreover, it has presently been discovered that the expression of such a variant YOR1 polypeptide in a yeast strain that is genetically modified to express one or more HMOs enhances production of the HMO(s) compared to a counterpart yeast strain that is genetically modified to express the one or more HMOs and the parental YOR1 polypeptide. Particularly, it has been discovered that expression of such a variant YOR1 polypeptide in a yeast cell genetically modified to biosynthesize one or more HMOs not only augments the overall yield of the HMO(s), but also improves the purity of the HMO(s) relative to a counterpart yeast strain modified to biosynthesize the HMO(s) but that lacks the variant YOR1 polypeptide or expresses the parental YOR1 polypeptide.

In one aspect of the invention, provided herein is a yeast cell genetically modified to produce one or more human milk oligosaccharides, wherein the yeast cell comprises (i) a heterologous nucleic acid encoding a variant human milk oligosaccharide (HMO) ABC transporter polypeptide having one or more amino acid substitutions compared to SEQ ID NO: 1; and (ii) one or more heterologous nucleic acids that each independently encode at least one enzyme of a human milk oligosaccharide biosynthetic pathway.

In an embodiment the amino acid substitution is selected from A446L, T220Q, I1127A, N1175Y, A256L, T220L, T226M, I373L, A446V, T1014G, Q64H, L795Q, and L1167S.

In another embodiment the variant ABC transporter polypeptide comprises an amino acid sequence selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

In a further embodiment the variant ABC transporter polypeptide comprises an amino acid sequence of SEQ ID NO: 2. In an embodiment the variant ABC transporter polypeptide comprises an amino acid sequence of SEQ ID NO: 3. In another embodiment the variant ABC transporter polypeptide comprises an amino acid sequence of SEQ ID NO: 4. In a further embodiment the variant ABC transporter polypeptide comprises an amino acid sequence of SEQ ID NO: 5. In yet another embodiment the variant ABC transporter polypeptide comprises an amino acid sequence of SEQ ID NO: 6. In other embodiments the variant ABC transporter polypeptide comprises an amino acid sequence of SEQ ID NO: 7. In other embodiments the variant ABC transporter polypeptide comprises an amino acid sequence of SEQ ID NO: 8. In other embodiments the variant ABC transporter polypeptide comprises an amino acid sequence of SEQ ID NO: 9. In other embodiments the variant ABC transporter polypeptide comprises an amino acid sequence of SEQ ID NO: 10. In other embodiments the variant ABC transporter polypeptide comprises an amino acid sequence of SEQ ID NO: 11. In other embodiments the variant ABC transporter polypeptide comprises an amino acid sequence of SEQ ID NO: 12. In other embodiments the variant ABC transporter polypeptide comprises an amino acid sequence of SEQ ID NO: 13. In other embodiments the variant ABC transporter polypeptide comprises an amino acid sequence of SEQ ID NO: 14. In another embodiment the variant ABC transporter polypeptide comprises an amino acid sequence of SEQ ID NO: 15.

In an embodiment the ABC transporter exports the human milk oligosaccharide 2′-fucosyllactose. In another embodiment the one or more human milk oligosaccharides comprise 2′-fucosyllactose. In another embodiment the heterologous nucleic acid encoding the ABC transporter polypeptide is integrated into the genome of the yeast cell and/or the one or more heterologous nucleic acids that each independently encode at least one enzyme of a human milk oligosaccharide biosynthetic pathway is integrated into the genome of the yeast cell. In an embodiment the yeast cell is a Saccharomyces sp. or a Kluveromyces sp. In a preferred embodiment the yeast cell is a Saccharomyces cerevisiae cell.

In another aspect the invention provides a method of producing one or more human milk oligosaccharides, the method comprising culturing a population of genetically modified yeast cells described herein in a culture medium under conditions suitable for the yeast cells to produce the one or more human milk oligosaccharides. In an embodiment the one or more human milk oligosaccharides comprise 2′-fucosyllactose.

In a further aspect the invention provides a fermentation composition comprising a population of genetically modified yeast cells comprising the yeast cells described herein, and a culture medium comprising one or more human milk oligosaccharides produced from the yeast cells. a yet another aspect the invention provides a method of recovering one or more human milk oligosaccharides from the fermentation composition described herein, the method comprising separating at least a portion of the population of genetically modified yeast cells from the culture medium; and contacting the separated yeast cells with a heated aqueous wash liquid.

### BRIEF DESCRIPTION OF THE DRAWINGS

FIG. 1 shows 2′-FL titers (Plate normal 2′-FL) vs. a ratio of 2′-FL titer to DFL titer (Plate normal 2′-FL:DFL) for each isolate normalized to a control strain on each plate. Promoted isolates for additional screening are shaped according to promotion criteria; squares (highest 2′-FL titer and increased 2′-FL:DFL ratio) circles (highest increase in total FL equivalents), or diamonds (increased 2′-FL titer and lowest lactose titer remaining).

FIG. 2 shows 2′-FL titers (Plate normal 2′-FL) vs. a ratio of 2′-FL titer to DFL titer (Plate normal 2′-FL:DFL) for each isolate normalized to a control strain on each plate. Vertical and horizontal error bars indicate standard deviation. Isolates are shaped by whether or not they were promoted for additional screening; square (promoted), circle and diamond (not promoted).

FIG. 3 shows 2′-FL titers (Normalized 2′-FL) normalized to the parent strain, Y77335 (black), of the wild-type YOR1 strain (gray) compared to different YOR1 variants (annotated and colored differently) screened in a 2′-FL producing strain. Boxes between two black vertical lines indicate YOR1 variant strains promoted for additional screening.

FIG. 4 is a set of two graphs comparing 2′-FL yield (top) and 2′-FL productivity (bottom) over time in fermentation in strains expressing the variant YOR1_A446L (strain Y77799) and wild-type YOR1 (strain Y77776).

FIG. 5 is a table showing the performance (2′-FL yield and productivity, normalized to a strain expressing wild-type YOR1, Y77776) of strains expressing ten distinct YOR1 variants (T220Q, I1127A, N1175Y, A446L. A256L, T220L, T266M, I373L, A446V, and T1014G) compared to a strain expressing wild-type YOR1 as determined in half-liter tank fermentations.

FIG. 6 is a set of three graphs showing 2′-FL yield (Normalized yield, top left) normalized to a strain expressing wild-type YOR1, 2′-FL productivity (Normalized productivity, top right) normalized to a strain expressing wild-type YOR1 and biomass (Normalized biomass) normalized to a strain expressing wild-type YOR1.

### DETAILED DESCRIPTION OF THE INVENTION

### Terminology

As used in the context of the present disclosure, “YOR1,” “Saccharomyces cerevisiae YOR1,” or “Sc.YOR1” is a human milk oligosaccharide ABC transporter polypeptide that has the ability to increase export of one or more HMOs produced by recombinant host cells that are engineered to express one or more enzymes of an HMO biosynthesis pathway. YOR1 has the amino acid sequence of SEQ ID NO: 1.

As used herein, “YOR1 variant” is a human milk oligosaccharide ABC transporter polypeptide that has one or more amino acid substitutions when compared to wild-type YOR1 (i.e., SEQ ID NO: 1). The YOR1 variants are indicated as YOR1_YxxxZ; wherein xxx is a number that represents the amino acid numbering of SEQ ID NO: 1, Y is the single letter code for the amino present in the wild-type YOR1 at position xxx of SEQ ID NO: 1, and Z is the single letter code for the amino acid present at position xxx of SEQ ID NO: 1 in the YOR1 variant polypeptide. Illustrative YOR1 variants include: YOR1_A446L; YOR1_T220Q; YOR1_I1127A; YOR1_N1175Y; YOR1_A256L; YOR1_T220L, YOR1_T266M, YOR1_I373L, YOR1_A446V, YOR1_T1014G, YOR1_Q64H, YOR1_L795Q, and YOR1_L1167S.

The terms “ABC transporter” and “ATP-binding cassette transporter” as used herein refer to proteins that are members of a large superfamily found in all kingdoms of life, which are responsible for the transport of compounds, such as drugs, ions, metabolites, lipids, vitamins, and organic compounds (e.g., HMOs), across cell membranes. ABC transporters that act as exporters can transport these compounds outward from the cytoplasm into the extracellular environment, while importers transport compounds into the cytoplasm.

The terms “human milk oligosaccharide” and “HMO” are used herein to refer to a group of nearly 200 identified sugar molecules that are found as the third most abundant component in human breast milk. HMOs in human breast milk are a complex mixture of free, indigestible carbohydrates with many different biological roles, including promoting the development of a functional infant immune system. HMOs include, without limitation, oligosaccharides that are fucosylated, such as 2′-fucosyllactose, 3-fucosyllactose, and difucosyllactose; galactosylated; sialylated; such as 3′-sialyllactose and 6′-sialyllactose; glycosylated; are neutral, such as lacto-N-tetraose and lacto-N-neotetraose; and may also include glucose, galactose, sialic acid, or N-acetylglucosamine.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA, or a hybrid, in which the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An exemplary algorithm that may be used to determine whether a polypeptide has sequence identity to any one of amino acid sequences disclosed herein is the BLAST algorithm, which is described in Altschul et al., 1990, J. Mol. Biol. 215:403-410, which is incorporated herein by reference. Software for performing BLAST analyses is publicly available through the National Center for Biotechnology Information (on the worldwide web at ncbi.nlm.nih.gov/). For amino acid sequences, the BLASTP program uses as defaults a word size (W) of 3, an expectation (E) of 10, and the BLOSUM62 scoring matrix (see Henikoff & Henikoff, 1989, Proc. Natl. Acad. Sci. USA 89:10915). Other programs that may be used include the Needleman-Wunsch procedure, J. Mol. Biol. 48: 443-453 (1970), using BLOSUM62, a Gap start penalty of 7 and gap extend penalty of 1; and gapped BLAST 2.0 (see Altschul, et al. 1997, Nucleic Acids Res., 25:3389-3402). Although various algorithms can be employed to determine percent identity, for purposes herein, % amino acid sequence identity values are generated using the sequence comparison computer program BLASTP (protein-protein BLAST algorithm) using default parameters.

Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following a sequence comparison algorithm or by manual alignment and visual inspection as described above. Optionally, the identity exists over a region that is at least about 50 nucleotides (or 20 amino acids) in length, or more preferably over a region that is 100 to 500 or 1000 or more nucleotides (or 50, 100, or 200 or more amino acids) in length.

Nucleic acid or protein sequences that are substantially identical to a reference sequence include “conservatively modified variants.” With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, at pH 7, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu, and Ile; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. The following six groups each contain amino acids that further provide illustrative conservative substitutions for one another. 1) Ala, Ser, Thr; 2) Asp, Glu; 3) Asn, Gln; 4) Arg, Lys; 5) Ile, Leu, Met, Val; and 6) Phe, Try, and Trp (see, e.g., Creighton, Proteins (1984)).

As used herein the term “heterologous” refers to what is not normally found in nature. The term “heterologous nucleic acid” refers to a nucleic acid not normally found in a given cell in nature. A heterologous nucleic acid can be: (a) foreign to its host cell, i.e., exogenous to the host cell such that a host cell does not naturally contain the nucleic acid; (b) naturally found in the host cell, i.e., endogenous or native to the host cell, but present at an unnatural quantity in the cell (i.e., greater or lesser quantity than naturally found in the host cell); (c) be naturally found in the host cell but positioned outside of its natural locus. A “heterologous” polypeptide refers to a polypeptide that is encoded by a “heterologous nucleic acid”. Thus, for example, a “heterologous” polypeptide may be naturally produced by a host cell but is encoded by a heterologous nucleic acid that has been introduced into the host cell by genetic engineering. For example, a “heterologous” polypeptide can include embodiments in which an endogenous polypeptide is produced by an expression construct and is overexpressed in the host cell compared to native levels of the polypeptide produced by the host cell.

As used herein, the term “introducing” in the context of introducing a nucleic acid or protein into a host cell refers to any process that results in the presence of a heterologous nucleic acid or polypeptide inside the host cell. For example, the term encompasses introducing a nucleic acid molecule (e.g., a plasmid or a linear nucleic acid) that encodes the nucleic acid of interest (e.g., an RNA molecule) or polypeptide of interest and results in the transcription of the RNA molecules and translation of the polypeptides. The term also encompasses integrating the nucleic acid encoding the RNA molecules or polypeptides into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein, the term “transformation” refers to a genetic alteration of a host cell resulting from the introduction of exogenous genetic material, e.g., nucleic acids, into the host cell.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

The term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As explained herein, these substantially identical variants are specifically covered by reference to a specific nucleic acid sequence. One example of an expression cassette is a polynucleotide construct that comprises a polynucleotide sequence encoding a polypeptide for use in the invention operably linked to a promoter, e.g., its native promoter, where the expression cassette is introduced into a heterologous microorganism. In some embodiments, an expression cassette comprises a polynucleotide sequence encoding a polypeptide of the invention where the polynucleotide that is targeted to a position in the genome of a microorganism such that expression of the polynucleotide sequence is driven by a promoter that is present in the microorganism.

The term “host cell” as used in the context of this invention refers to a microorganism, such as yeast, and includes an individual cell or cell culture comprising a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “promoter” refers to a nucleic acid control sequences that can direct transcription of a nucleic acid. A promoter includes necessary nucleic acid sequences near the start site of transcription. A promoter also optionally includes distal enhancer or repressor elements, which can be located as much as several thousand base pairs from the start site of transcription.

As used herein, the term “genetic switch” refers to one or more genetic elements that allow controlled expression of enzymes, e.g., enzymes that catalyze the reactions of human milk oligosaccharide biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “operably linked” refers to a functional linkage between nucleic acid sequences such that the sequences encode a desired function. For example, a coding sequence for a gene of interest, e.g., a variant YOR1 polypeptide, is in operable linkage with its promoter and/or regulatory sequences when the linked promoter and/or regulatory region functionally controls expression of the coding sequence. It also refers to the linkage between coding sequences such that they may be controlled by the same linked promoter and/or regulatory region; such linkage between coding sequences may also be referred to as being linked in frame or in the same coding frame. “Operably linked” also refers to a linkage of functional but non-coding sequences, such as an autonomous propagation sequence or origin of replication. Such sequences are in operable linkage when they are able to perform their normal function, e.g., enabling the replication, propagation, and/or segregation of a vector bearing the sequence in a host cell.

The term “enhanced” in the context of increased production of one or more HMOs from a genetically modified host cell as described herein refers to an increase in the production of at least one HMO by a host cell genetically modified to express a YOR1 variant described herein, in comparison to a control counterpart host cell that produced the at least one HMO, but does not have the genetic modification to expression the ABC transporter. Production of at least one HMO is typically enhanced by at least 5%, or at least 10%, 20%, 30%, 40%, 50%, 60%, 70%, 80%, 90%, 100%, or greater compared to the control cell.

As used herein with respect to expression of a non-native ABC transporter polypeptide in a host cell that does not naturally express the YOR1 variant, the terms “expression” and “overexpression” are used interchangeably.

As used in this specification and the appended claims, the singular forms “a,” “an,” and “the” include plural reference unless the context clearly dictates otherwise.

As used herein, the term “about” is used herein to mean a value that is ±10% of the recited value.

### Modifications to Host Cells to Enhance Production of One or More HMOs

### Overview

### ABC Transporters

ATP binding cassette (ABC) transporter polypeptides, referred to as “ABC” transporters, are widespread in all forms of life and are characterized by two nucleotide-binding domains (NBD) and two transmembrane domains (TMDs). ABC transporters function to transport compounds such as drugs, ions, metabolites, lipids, vitamins, and organic compounds across a cell membrane. Without being limited by mechanism or theory, transport is generally driven by ATP hydrolysis on the NBD, causing conformational changes in the TMD. This results in alternating access from inside and outside of the cell for unidirectional transport across the lipid bilayer. Common to all ABC transporters is a signature sequence or motif, LSGGQ, that is involved in nucleotide binding. The majority of eukaryotic ABC transporter family members function in the direction of exporting compounds from the cytoplasmic side of the membrane outward. As a result, ABC transporters may be heterologously expressed to export compounds from a cell, such as a yeast cell. X-ray crystal structure determination of a variety of bacterial and eukaryotic ABC transporters has advanced understanding of the ATP hydrolysis-driven transport mechanism.

### Human Milk Oligosaccharide (HMO) ABC Transporters

ABC transporters may exhibit substrate specificity, acting primarily on one particular substrate or a structural variant thereof. The substrate specificity of an ABC transporter is dictated by the structure and amino acid sequence of the ABC transporter. It has presently been discovered that some ABC transporters are able to export HMOs across cell membranes. Thus, the present disclosure provides ABC transporters that have now been discovered to have HMO transporter properties. The ABC transporters provided herein give rise to beneficial biosynthetic properties, as these transporters have been presently discovered to not only engender heightened HMO production, but also improved HMO product purity. Thus, the ABC transporters provided herein may be heterologously expressed in yeast cells to increase export of one or more HMOs produced by recombinant yeast cells that are engineered to express one or more enzymes of a HMO biosynthesis pathway.

Saccharomyces cerevisiae YOR1 is an ABC transporter that has been shown to export HMOs from host cells genetically engineered to produce the HMOs and thereby increase the productivity and yield of HMO production. Illustrative variant YOR1 polypeptides described herein, including YOR1_A446L; YOR1_T220Q; YOR1_I1127A; YOR1_N1175Y; YOR1_A256L; YOR1_T220L, YOR1_T266M, YOR1_I373L, YOR1_A446V, YOR1_T1014G, YOR1_Q64H, YOR1_L795Q, YOR1_L1167S, and YOR1_F333A (SEQ ID NO: 2, SEQ ID NO: 3, SEQ ID NO: 4, SEQ ID NO: 5, SEQ ID NO: 6, SEQ ID NO:7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15) have increased HMO export potential and increase HMO production when expressed in HMO producing host cells compared to HMO producing host cells expressing wild-type YOR1.

In some embodiments, a yeast host cell is genetically modified in accordance with the invention to express a variant YOR1 polypeptide having an amino acid sequence of any one of SEQ ID NOS: 2-15, or a biologically active variant that shares substantial identity with any one of SEQ ID NOS: 2-15. In some embodiments, the variant has at least 70%, or at least 75%, 80%, or 85% identity to any one of SEQ ID NOS: 2-15. In some embodiments, the variant has at least 90%, or at least 91%, 92%, 93%, or 94% identity to the amino acid sequence of any one of SEQ ID NOS: 2-15. In some embodiments, the variant has at least 95% identity to any one of SEQ ID NOS: 2-15. As used herein, the term “variant” encompasses biologically active polypeptides having one or more substitutions, deletions, or insertions relative to any one of SEQ ID NOS: 2-15. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 2, or a biologically active variant that shares substantial identity with SEQ ID NO: 2. In some embodiments, the variant has at least 70%, or at least 75%, 80%, or 85% identity to SEQ ID NO: 2. In some embodiments, the variant has at least 90%, or at least 91%, 92%, 93%, or 94% identity to the amino acid sequence of SEQ ID NO: 2. In some embodiments, the variant has at least 95% identity to SEQ ID NO: 2. As used herein, the term “variant” encompasses biologically active polypeptides having one or more substitutions, deletions, or insertions relative to SEQ ID NO: 2. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 3, or a biologically active variant that shares substantial identity with SEQ ID NO: 3. In some embodiments, the variant has at least 70%, or at least 75%, 80%, or 85% identity to SEQ ID NO: 3. In some embodiments, the variant has at least 90%, or at least 91%, 92%, 93%, or 94% identity to the amino acid sequence of SEQ ID NO: 3. In some embodiments, the variant has at least 95% identity to SEQ ID NO:3. As used herein, the term “variant” encompasses biologically active polypeptides having one or more substitutions, deletions, or insertions relative to SEQ ID NO: 3. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 4, or a biologically active variant that shares substantial identity with SEQ ID NO: 4. In some embodiments, the variant has at least 70%, or at least 75%, 80%, or 85% identity to SEQ ID NO: 4. In some embodiments, the variant has at least 90%, or at least 91%, 92%, 93%, or 94% identity to the amino acid sequence of SEQ ID NO: 4. In some embodiments, the variant has at least 95% identity to SEQ ID NO: 4. As used herein, the term “variant” encompasses biologically active polypeptides having one or more substitutions, deletions, or insertions relative to SEQ ID NO: 4. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 5, or a biologically active variant that shares substantial identity with SEQ ID NO: 5. In some embodiments, the variant has at least 70%, or at least 75%, 80%, or 85% identity to SEQ ID NO: 5. In some embodiments, the variant has at least 90%, or at least 91%, 92%, 93%, or 94% identity to the amino acid sequence of SEQ ID NO: 5. In some embodiments, the variant has at least 95% identity to SEQ ID NO: 5. As used herein, the term “variant” encompasses biologically active polypeptides having one or more substitutions, deletions, or insertions relative to SEQ ID NO: 5. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 6, or a biologically active variant that shares substantial identity with SEQ ID NO: 6. In some embodiments, the variant has at least 70%, or at least 75%, 80%, or 85% identity to SEQ ID NO: 6. In some embodiments, the variant has at least 90%, or at least 91%, 92%, 93%, or 94% identity to the amino acid sequence of SEQ ID NO: 6. In some embodiments, the variant has at least 95% identity to SEQ ID NO: 6. As used herein, the term “variant” encompasses biologically active polypeptides having one or more substitutions, deletions, or insertions relative to SEQ ID NO: 6. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 7, or a biologically active variant that shares substantial identity with SEQ ID NO: 7. In some embodiments, the variant has at least 70%, or at least 75%, 80%, or 85% identity to SEQ ID NO: 7. In some embodiments, the variant has at least 90%, or at least 91%, 92%, 93%, or 94% identity to the amino acid sequence of SEQ ID NO: 7. In some embodiments, the variant has at least 95% identity to SEQ ID NO: 7. As used herein, the term “variant” encompasses biologically active polypeptides having one or more substitutions, deletions, or insertions relative to SEQ ID NO: 7. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 8, or a biologically active variant that shares substantial identity with SEQ ID NO: 8. In some embodiments, the variant has at least 70%, or at least 75%, 80%, or 85% identity to SEQ ID NO: 8. In some embodiments, the variant has at least 90%, or at least 91%, 92%, 93%, or 94% identity to the amino acid sequence of SEQ ID NO: 8. In some embodiments, the variant has at least 95% identity to SEQ ID NO: 8. As used herein, the term “variant” encompasses biologically active polypeptides having one or more substitutions, deletions, or insertions relative to SEQ ID NO: 8. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 8, or a biologically active variant that shares substantial identity with SEQ ID NO: 8. In some embodiments, the variant has at least 70%, or at least 75%, 80%, or 85% identity to SEQ ID NO: 8. In some embodiments, the variant has at least 90%, or at least 91%, 92%, 93%, or 94% identity to the amino acid sequence of SEQ ID NO: 8. In some embodiments, the variant has at least 95% identity to SEQ ID NO: 8. As used herein, the term “variant” encompasses biologically active polypeptides having one or more substitutions, deletions, or insertions relative to SEQ ID NO: 8. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 9, or a biologically active variant that shares substantial identity with SEQ ID NO: 9. In some embodiments, the variant has at least 70%, or at least 75%, 80%, or 85% identity to SEQ ID NO: 9. In some embodiments, the variant has at least 90%, or at least 91%, 92%, 93%, or 94% identity to the amino acid sequence of SEQ ID NO: 9. In some embodiments, the variant has at least 95% identity to SEQ ID NO: 9. As used herein, the term “variant” encompasses biologically active polypeptides having one or more substitutions, deletions, or insertions relative to SEQ ID NO: 9. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 10, or a biologically active variant that shares substantial identity with SEQ ID NO: 10. In some embodiments, the variant has at least 70%, or at least 75%, 80%, or 85% identity to SEQ ID NO: 10. In some embodiments, the variant has at least 90%, or at least 91%, 92%, 93%, or 94% identity to the amino acid sequence of SEQ ID NO: 10. In some embodiments, the variant has at least 95% identity to SEQ ID NO: 10. As used herein, the term “variant” encompasses biologically active polypeptides having one or more substitutions, deletions, or insertions relative to SEQ ID NO: 10. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 11, or a biologically active variant that shares substantial identity with SEQ ID NO: 11. In some embodiments, the variant has at least 70%, or at least 75%, 80%, or 85% identity to SEQ ID NO: 11. In some embodiments, the variant has at least 90%, or at least 91%, 92%, 93%, or 94% identity to the amino acid sequence of SEQ ID NO: 11. In some embodiments, the variant has at least 95% identity to SEQ ID NO: 11. As used herein, the term “variant” encompasses biologically active polypeptides having one or more substitutions, deletions, or insertions relative to SEQ ID NO: 11. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 12, or a biologically active variant that shares substantial identity with SEQ ID NO: 12. In some embodiments, the variant has at least 70%, or at least 75%, 80%, or 85% identity to SEQ ID NO: 12. In some embodiments, the variant has at least 90%, or at least 91%, 92%, 93%, or 94% identity to the amino acid sequence of SEQ ID NO: 12. In some embodiments, the variant has at least 95% identity to SEQ ID NO: 12. As used herein, the term “variant” encompasses biologically active polypeptides having one or more substitutions, deletions, or insertions relative to SEQ ID NO: 12. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 13, or a biologically active variant that shares substantial identity with SEQ ID NO: 13. In some embodiments, the variant has at least 70%, or at least 75%, 80%, or 85% identity to SEQ ID NO: 13. In some embodiments, the variant has at least 90%, or at least 91%, 92%, 93%, or 94% identity to the amino acid sequence of SEQ ID NO: 13. In some embodiments, the variant has at least 95% identity to SEQ ID NO: 13. As used herein, the term “variant” encompasses biologically active polypeptides having one or more substitutions, deletions, or insertions relative to SEQ ID NO: 13. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 14, or a biologically active variant that shares substantial identity with SEQ ID NO: 14. In some embodiments, the variant has at least 70%, or at least 75%, 80%, or 85% identity to SEQ ID NO: 14. In some embodiments, the variant has at least 90%, or at least 91%, 92%, 93%, or 94% identity to the amino acid sequence of SEQ ID NO: 14. In some embodiments, the variant has at least 95% identity to SEQ ID NO: 14. As used herein, the term “variant” encompasses biologically active polypeptides having one or more substitutions, deletions, or insertions relative to SEQ ID NO: 14. Thus, the term “variant” includes biologically active fragments as well as substitution variants.

In some embodiments, a yeast host cell is genetically modified in accordance with the invention to express an ABC transporter polypeptide having the amino acid sequence of SEQ ID NO: 15, or a biologically active variant that shares substantial identity with SEQ ID NO: 15. In some embodiments, the variant has at least 70%, or at least 75%, 80%, or 85% identity to SEQ ID NO: 15. In some embodiments, the variant has at least 90%, or at least 91%, 92%, 93%, or 94% identity to the amino acid sequence of SEQ ID NO: 15. In some embodiments, the variant has at least 95% identity to SEQ ID NO: 15. As used herein, the term “variant” encompasses biologically active polypeptides having one or more substitutions, deletions, or insertions relative to SEQ ID NO: 15. Thus, the term “variant” includes biologically active fragments as well as substitution variants.

ABC transporter activity can be assessed using any number of assays, including assays that evaluate the overall production of at least one HMO by a yeast cell strain. For example, production yields are calculated by quantifying sugar input into fermentation tanks and measuring residual sucrose levels and constituent glucose and fructose monomers, via comparison to known standard concentrations and analysis through ion exchange chromatography. Thus, for example, yield of 2′-FL is therefore assessed by comparing 2′-FL output to sucrose input. In some embodiments, the production yield of 2′-fucosyllactose by a genetically modified yeast strain is measured by quantifying total sucrose fed and total 2′-fucosyllactose produced using ion exchange chromatography (IC). Yield is reported as g 2′-fucosyllactose/g sucrose. Any other method that allows one of skill to assess ABC transporter activity may also be employed.

### Yeast Genetically Modified to Produce HMO

Provided herein are genetically modified yeast cells capable of producing one or more HMOs, which yeast cells are further modified to express a heterologous ABC transport polypeptide, e.g., SEQ ID NOS: 2-15, or a biologically active variant thereof. Such yeast cells include one or more heterologous nucleic acids, each independently encoding an enzyme of a HMO biosynthetic pathway; and a heterologous nucleic acid encoding an export protein, e.g., an ABC transporter such as a polypeptide comprising the amino acid of any one of SEQ ID NOS: 2-15; and any other one of SEQ ID NOS: 2-15; or a variant thereof, that mediates export of an HMO. In some embodiments, the biosynthetic pathways of the provided yeast cells generate GDP-fucose from an external sugar such as glucose or sucrose, and not from external fucose.

In some embodiments, the provided genetically modified yeast cells are capable of producing the UDP-glucose HMO precursor. The activated sugar UDP-glucose is composed of a pyrophosphate group, the pentose sugar ribose, glucose, and the nucleobase uracil. UDP-glucose is natively produced by yeast cells, and its production levels can be increased with overexpression of, for example, phosphoglucomutase-2 (PGM2) or UTP glucose-1-phosphate uridylyltransferase (UGP1).

In some embodiment, the provided genetically modified yeast cells are capable of producing the UDP-galactose HMO precursor. The activated sugar UDP-galactose is composed of a pyrophosphate group, the pentose sugar ribose, galactose, and the nucleobase uracil. UDP-galactose is natively produced by yeast cells, and its production levels can be increased with overexpression of, for example, UDP-glucose-4-epimerase (GAL10).

In some embodiments, the provided genetically modified yeast cells are capable of producing the UDP-N-acetylglucosamine HMO precursor. The activated sugar UDP-N-acetylglucosamine consists of a pyrophosphate group, the pentose sugar ribose, N-acetylglucosamine, and the nucleobase uracil. UDP-N-acetylglucosamine is natively produced by yeast cells, and its production levels can be increased with expression of, for example, UDP-N-acetylglucosamine-diphosphorylase, or overexpression of, for example, glucosamine 6-phosphate N-acetyltransferase (GNA1) or phosphoacetylglucosamine mutase (PCM1).

In some embodiments, the provided genetically modified yeast cells are capable of producing the GDP-fucose HMO precursor. The activated sugar GDP-fucose consists of a pyrophosphate group, the pentose sugar ribose, fucose, and the nucleobase guanine. GDP-fucose is not natively produced by yeast cells, and its production can be enabled with the introduction of, for example, GDP-mannose 4,6-dehydratase, e.g., from Escherichia coli, and GDP-L-fucose synthase, e.g., from Arabidopsis thaliana.

In some embodiments, the provided genetically modified yeast cells are capable of producing the CMP-sialic acid HMO precursor. The activated sugar CMP-sialic acid consists of a pyrophosphate group, the pentose sugar ribose, sialic acid, and the nucleobase cytosine. CMP-sialic acid is not natively produced by yeast cells, and its production can be enabled with the introduction of, for example, CMP-Neu5Ac synthetase, e.g., from Campylobacter jejuni, sialic acid synthase, e.g., from C. jejuni, and UDP-N-acetylglucosamine 2-epimerase, e.g., from C. jejuni.

In some embodiments, the genetically modified yeast is capable of producing 2′-fucosyllactose. In addition to one or more heterologous nucleic acids encoding one or more of the aforementioned enzymes, the yeast can further include one or more heterologous nucleic acids encoding one or more of GDP-mannose 4,6-dehydratase, e.g., from Escherichia coli, GDP-L-fucose synthase, e.g., from Arabidopsis thaliana, a-1,2-fucosyltransferase, e.g., from Helicobacter pylori, and a fucosidase, e.g., an α-1,3-fucosidase. In some embodiments, the fucosyltransferase is from Candidata moranbacterium or Pseudoalteromonas haloplanktis.

In some embodiments, the genetically modified yeast cell includes a heterologous nucleic acid encoding an enzyme that can catalyze the conversion of GDP-mannose to GDP-4-dehydro-6-deoxy-D-mannose, e.g., a GDP-mannose 4,6-dehydratase. In some embodiments, the GDP-mannose 4,6-dehydratase is from Escherichia coli. Other suitable GDP-mannose 4,6-dehydratase sources include, for example and without limitation, Caenorhabditis elegans, Homo sapiens, Arabidopsis thaliana, Dictyostelium discoideum, Mus musculus, Drosophila melanogaster, Sinorhizobium fredii HH 103, Sinorhizobium fredii NGR234, Planctomycetes bacterium RBG_13_63_9, Silicibacter sp. TrichCH4B, Pandoraea vervacti, Bradyrhizobium sp. YR681, Epulopiscium sp. SCG-B11WGA-EpuloA1, Caenorhabditis briggsae, Candidatus Curtissbacteria bacterium RIFCSPLOWO2_12_FULL_38_9, Pseudomonas sp. EpS/L25, Clostridium sp. KLE 1755, mine drainage metagenome, Nitrospira sp. SG-bin2, Cricetulus griseus, Arthrobacter siccitolerans, and Paraburkholderia piptadeniae. In some embodiments, the GDP-mannose dehydratase is from Caenorhabditis briggsae or Escherichia coli.

In some embodiments, the genetically modified yeast cell includes a heterologous nucleic acid encoding an enzyme that can catalyze the conversion of GDP-4-dehydro-6-deoxy-D-mannose to GDP-L-fucose, e.g., a GDP-L-fucose synthase. In some embodiments, the GDP-L-fucose synthase is from Arabidopsis thaliana. Other suitable GDP-L-fucose synthase sources include, for example and without limitation, Mus musculus, Escherichia coli K-12, Homo sapiens, Marinobacter salarius, Sinorhizobium fredii NGR234, Oryza sativa Japonica Group, Micavibrio aeruginosavorus ARL-13, Citrobacter sp. 86, Pongo abelii, Caenorhabditis elegans, Candidatus Staskawiczbacteria bacterium RIFCSPHIGHO2_01_FULL_41_41, Drosophila melanogaster, Azorhizobium caulinodans ORS 571, Candidatus Nitrospira nitrificans, Mycobacterium elephantis, Elusimicrobia bacterium RBG_16_66_12, Vibrio sp. JCM 19231, Planktothrix serta PCC 8927, Thermodesulfovibrio sp. RBG_19FT_COMBO_42_12, Anaerovibrio sp. JC8, Dictyostelium discoideum, and Cricetulus griseus.

In some embodiments, the genetically modified yeast cell includes a heterologous nucleic acid encoding an enzyme that can catalyze the conversion of GDP-L-fucose and lactose to 2′-fucosyllactose, e.g., an α-1,2-fucosyltransferase. In some embodiments, the α-1,2-fucosyltransferase is from Helicobacter pylori. In some embodiments, the fucosyltransferase is from Candidata moranbacterium or Pseudoalteromonas haloplanktis ANT/505. Other suitable α-1,2-fucosyltransferase sources include, for example and without limitation, Escherichia coli, Sus scrofa, Homo sapiens, Chlorocebus sabaeus, Pan troglodytes, Gorilla gorilla gorilla, Macaca mulatta, Oryctolagus cuniculus, Pongo pygmaeus, Mus musculus, Rattus norvegicus, Caenorhabditis elegans, Hylobates lar, Bos taurus, Hylobates agilis, Eulemur fulvus, and Helicobacter hepaticus ATCC 51449. In some embodiments, the source of the α-1,2-fucosyltransferase is Pseudoalteromonas haloplanktis ANT/505, Candidatus moranbacteria bacterium, Acetobacter sp. CAG: 267, Bacteroides vulgatus, Sulfurovum lithotrophicum, Thermosynechococcus elongatus BP-1, Geobacter uraniireducens Rf4, Bacteroides fragilis str. S23L17, Chromobacterium vaccinii, Herbaspirillum sp. YR522, or Helicobacter bilis ATCC 43879.

In some embodiments, the genetically modified yeast cell includes a heterologous nucleic acid encoding an enzyme that can catalyze the conversion of difucosyllactose to 2′-fucosyllactose and fucose, e.g., an α1-3,4-fucosidase. Suitable α1-3,4-fucosidase sources include, for example and without limitations, Bacteroides thetaiotaomicron, Bifidobacterium bifidum, Bifidobacterium longum, Bifidobacterium longum subsp. infantis, Clostridium perfringens, Lactobacillus casei, Paenibacillus thiaminolyticus, Pseudomonas putida, Thermotoga maritima, Xanthomonas campestris pv. campestris, Arabidopsis thaliana, and Rattus norvegicus.

In some embodiments, the genetically modified yeast is capable of producing 3-fucosyllactose. In addition to one or more heterologous nucleic acids encoding one or more of the aforementioned enzymes, the yeast can further include one or more heterologous nucleic acids encoding one or more of GDP-mannose 4,6-dehydratase, e.g., from Escherichia coli, GDP-L-fucose synthase, e.g., from Arabidopsis thaliana, α-1,3-fucosyltransferase, e.g., from Helicobacter pylori, and a fucosidase, e.g., an α-1,2-fucosidase.

In some embodiments, the genetically modified yeast cell includes a heterologous nucleic acid encoding an enzyme that can catalyze the conversion of GDP-L-fucose and lactose to 3-fucosyllactose, e.g., an α-1,3-fucosyltransferase. In some embodiments, the α-1,3-fucosyltransferase is from Helicobacter pylori. Other suitable α-1,3-fucosyltransferase sources include, for example and without limitation, Homo sapiens, Escherichia coli, Sus scrofa, Chlorocebus sabaeus, Pan troglodytes, Gorilla gorilla gorilla, Macaca mulatta, Oryctolagus cuniculus, Pongo pygmaeus, Mus musculus, Rattus norvegicus, Caenorhabditis elegans, Hylobates lar, Bos taurus, Hylobates agilis, Eulemur fulvus, Helicobacter hepaticus ATCC 51449, Akkermansia muciniphila, Bacteroides fragilis, and Zea mays.

In some embodiments, the genetically modified yeast is capable of producing lacto-N-tetraose. In addition to one or more heterologous nucleic acids encoding one or more of the aforementioned enzymes, the yeast can further include one or more heterologous nucleic acids encoding one or more of β-1,3-N-acetylglucosaminyltransferase, e.g., from Neisseria meningitidis, β-1,3-galactosyltransferase, e.g., from Escherichia coli, and UDP-N-acetylglucosamine-diphosphorylase, e.g., from E. coli.

In some embodiments, the genetically modified yeast cell includes a heterologous nucleic acid encoding an enzyme that can catalyze the conversion of UDP-N-acetyl-alpha-D-glucosamine and lactose to lacto-N-triose II and UDP, e.g., a β-1,3-N-acetylglucosaminyltransferase. In some embodiments, the β-1,3-N-acetylglucosaminyltransferase is from Neisseria meningitidis. Other suitable β-1,3-N-acetylglucosaminyltransferase sources include, for example and without limitation, Arabidopsis thaliana, Streptococcus dysgalactiae sub sp. equisimilis, Escherichia coli, e.g., Escherichia coli K-12, Pseudomonas aeruginosa PAO1, Homo sapiens, Mus musculus, Mycobacterium smegmatis str. MC2 155, Dictyostelium discoideum, Komagataeibacter hansenii, Aspergillus nidulans FGSC A4, Schizosaccharomyces pombe 972h-, Neurospora crassa OR74A, Aspergillus fumigatus Af293, Ustilago maydis 521, Bacillus subtilis subsp. subtilis str. 168, Rattus norvegicus, Listeria monocytogenes EGD-e, Bradyrhizobium japonicum, Nostoc sp. PCC 7120, Haloferax volcanii DS2, Caulobacter crescentus CB15, Mycobacterium avium subsp. silvaticum, Oenococcus oeni, Neisseria gonorrhoeae, Propionibacterium freudenreichii subsp. shermanii, Escherichia coli O157:H7, Aggregatibacter actinomycetemcomitans, Bradyrhizobium diazoefficiens USDA 110, Francisella tularensis subsp. novicida U112, Komagataeibacter xylinus, Haemophilus influenzae Rd KW20, Fusobacterium nucleatum subsp. nucleatum ATCC 25586, Bacillus phage SPbeta, Coccidioides posadasii, Populus tremula×Populus alba, Rhizopus microsporus var. oligosporus, Streptococcus parasanguinis, Shigella flexneri, Caenorhabditis elegans, Hordeum vulgare, Synechocystis sp. PCC 6803 substr. Kazusa, Streptococcus agalactiae, Plasmopara viticola, Staphylococcus epidermidis RP62A, Shigella phage SfII, Plasmid pWQ799, Fusarium graminearum, Sinorhizobium meliloti 1021, Physcomitrella patens, Sphingomonas sp. S88, Streptomyces hygroscopicus sub sp. jinggangensis 5008, Drosophila melanogaster, Phytophthora infestans, Staphylococcus aureus subsp. aureus Mu50, Penicillium chrysogenum, and Tribolium castaneum.

In some embodiments, the genetically modified yeast cell includes a heterologous nucleic acid encoding an enzyme that can catalyze the conversion of UDP-galactose and lacto-N-triose II to lacto-N-tetraose and UDP, e.g., a β-1,3-galactosyltransferase. In some embodiments, the β-1,3-galactosyltransferase is from Escherichia coli. Other suitable β-1,3-galactosyltransferase sources include, for example and without limitation, Arabidopsis thaliana, Streptococcus dysgalactiae subsp. equisimilis, Pseudomonas aeruginosa PAO1, Homo sapiens, Mus musculus, Mycobacterium smegmatis str. MC2 155, Dictyostelium discoideum, Komagataeibacter hansenii, Aspergillus nidulans FGSC A4, Schizosaccharomyces pombe 972h-, Neurospora crassa OR74A, Aspergillus fumigatus Af293, Ustilago maydis 521, Bacillus subtilis subsp. subtilis str. 168, Rattus norvegicus, Neisseria meningitidis, Listeria monocytogenes EGD-e, Bradyrhizobium japonicum, Nostoc sp. PCC 7120, Haloferax volcanii DS2, Caulobacter crescentus CB15, Mycobacterium avium subsp. silvaticum, Oenococcus oeni, Neisseria gonorrhoeae, Propionibacterium freudenreichii subsp. shermanii, Aggregatibacter actinomycetemcomitans, Bradyrhizobium diazoefficiens USDA 110, Francisella tularensis subsp. novicida U112, Komagataeibacter xylinus, Haemophilus influenzae Rd KW20, Fusobacterium nucleatum subsp. nucleatum ATCC 25586, Bacillus phage SPbeta, Coccidioides posadasii, Populus tremula×Populus alba, Rhizopus microsporus var. oligosporus, Streptococcus parasanguinis, Shigella flexneri, Caenorhabditis elegans, Hordeum vulgare, Synechocystis sp. PCC 6803 substr. Kazusa, Streptococcus agalactiae, Plasmopara viticola, Staphylococcus epidermidis RP62A, Shigella phage SfII, Plasmid pWQ799, Fusarium graminearum, Sinorhizobium meliloti 1021, Physcomitrella patens, Sphingomonas sp. S88, Streptomyces hygroscopicus subsp. jinggangensis 5008, Drosophila melanogaster, Phytophthora infestans, Staphylococcus aureus subsp. aureus Mu50, Penicillium chrysogenum, and Tribolium castaneum.

In some embodiments, the genetically modified yeast cell includes a heterologous nucleic acid encoding an enzyme that can catalyze the conversion of N-acetyl-α-D-glucosamine 1-phosphate to UDP-N-acetyl-α-D-glucosamine, e.g., a UDP-N-acetylglucosamine-diphosphorylase. In some embodiments, the UDP-N-acetylglucosamine-diphosphorylase is from Escherichia coli.

In some embodiments, the genetically modified yeast is capable of producing lacto-N-neotetraose. In addition to one or more heterologous nucleic acids encoding one or more of the aforementioned enzymes, the yeast can further include one or more heterologous nucleic acids encoding one or more of β-1,3-N-acetylglucosaminyltransferase, e.g., from Neisseria meningitidis, p-1,4-galactosyltransferase, e.g., from N. meningitidis, and UDP-N-acetylglucosamine-diphosphorylase, e.g., from E. coli.

In some embodiments, the genetically modified yeast cell includes a heterologous nucleic acid encoding an enzyme that can catalyze the conversion of UDP-galactose and lacto-N-triose II to lacto N-neotetraose and UDP, e.g., a β-1,4-galactosyltransferase. In some embodiments, the β-1,4-galactosyltransferase is from Neisseria meningitidis. Other suitable β-1,4-galactosyltransferase sources include, for example and without limitation, Homo sapiens, Neisseria gonorrhoeae, Haemophilus influenzae, Acanthamoeba polyphaga mimivirus, Haemophilus influenzae Rd KW20, Haemophilus ducreyi 35000HP, Moraxella catarrhalis, [Haemophilus] ducreyi, Aeromonas salmonicida subsp. salmonicida A449, and Helicobacter pylori 26695.

In some embodiments, the genetically modified yeast is capable of producing 3′-sialyllactose. In addition to heterologous nucleic acids encoding one or more of the aforementioned enzymes, the yeast can further include heterologous nucleic acids encoding CMP-Neu5Ac synthetase, e.g., from Campylobacter jejuni, sialic acid synthase, e.g., from C. jejuni, UDP-N-acetylglucosamine 2-epimerase, e.g., from C. jejuni, UDP-N-acetylglucosamine-diphosphorylase, e.g., from E. coli, and CMP-N-acetylneuraminate-β-galactosamide-α-2,3-sialyltransferase, e.g., from N. meningitides MC58.

In some embodiments, the genetically modified yeast cell includes a heterologous nucleic acid encoding an enzyme that can catalyze the conversion of UDP-N-acetyl-α-D-glucosamine to N-acetyl-mannosamine and UDP, e.g., a UDP-N-acetylglucosamine 2-epimerase. In some embodiments, the UDP-N-acetylglucosamine 2-epimerase is from Campylobacter jejuni. Other suitable UDP-N-acetylglucosamine 2-epimerase sources include, for example and without limitation, Homo sapiens, Rattus norvegicus, Mus musculus, Dictyostelium discoideum, Plesiomonas shigelloides, Bacillus subtilis subsp. subtilis str. 168, Bacteroides fragilis, Geobacillus kaustophilus HTA426, Synechococcus sp. CC9311, Sphingopyxis alaskensis RB2256, Synechococcus sp. RS9916, Moorella thermoacetica ATCC 39073, Psychrobacter sp. 1501(2011), Zunongwangia profunda SM-A87, Thiomicrospira crunogena XCL-2, Polaribacter sp. MED152, Vibrio campbellii ATCC BAA-1116, Thiomonas arsenitoxydans, Nitrobacter winogradskyi Nb-255, Raphidiopsis brookii D9, Thermoanaerobacter italicus Ab9, Roseobacter litoralis Och 149, Halothiobacillus neapolitanus c2, Halothiobacillus neapolitanus c2, Bacteroides vulgatus ATCC 8482, Zunongwangia profunda SM-A87, Moorella thermoacetica ATCC 39073, Paenibacillus polymyxa E681, Desulfatibacillum alkenivorans AK-01, Magnetospirillum magneticum AMB-1, Thermoanaerobacter italicus Ab9, Paenibacillus polymyxa E681, Prochlorococcus marinus str. MIT 9211, Subdoligranulum variabile DSM 15176, Kordia algicida OT-1, Bizionia argentinensis JUB59, Tannerella forsythia 92A2, Thiomonas arsenitoxydans, Synechococcus sp. BL107, Escherichia coli, Vibrio campbellii ATCC BAA-1116, Rhodopseudomonas palustris HaA2, Roseobacter litoralis Och 149, Synechococcus sp. CC9311, Subdoligranulum variabile DSM 15176, Bizionia argentinensis JUB59, Selenomonas sp. oral taxon 149 str. 67H29BP, Bacteroides vulgatus ATCC 8482, Kordia algicida OT-1, Desulfatibacillum alkenivorans AK-01, Thermodesulfovibrio yellowstonii DSM 11347, Desulfovibrio aespoeensis Aspo-2, Synechococcus sp. BL107, and Desulfovibrio aespoeensis Aspo-2.

In some embodiments, the genetically modified yeast cell includes a heterologous nucleic acid encoding an enzyme that can catalyze the conversion of N-acetyl-mannosamine and phosphoenolpyruvate to N-acetylneuraminate, e.g., a sialic acid synthase. In some embodiments, the sialic acid synthase is from Campylobacter jejuni. Other suitable sialic acid synthase sources include, for example and without limitation, Homo sapiens, groundwater metagenome, Prochlorococcus marinus str. MIT 9211, Rhodospirillum centenum SW, Rhodobacter capsulatus SB 1003, Aminomonas paucivorans DSM 12260, Ictalurus punctatus, Octadecabacter antarcticus 307, Octadecabacter arcticus 238, Butyrivibrio proteoclasticus B316, Neisseria meningitidis serogroup B., Idiomarina loihiensis L2TR, Butyrivibrio proteoclasticus B316, and Campylobacter jejuni.

In some embodiments, the genetically modified yeast cell includes a heterologous nucleic acid encoding an enzyme that can catalyze the conversion of N-acetylneuraminate and CTP to CMP-N-acetylneuraminate, e.g., a CMP-Neu5Ac synthetase. In some embodiments, the CMP-Neu5Ac synthetase is from Campylobacter jejuni. Other suitable CMP-Neu5Ac synthetase sources include, for example and without limitation, Neisseria meningitidis, Streptococcus agalactiae NEM316, Homo sapiens, Mus musculus, Bacteroides thetaiotaomicron, Pongo abelii, Danio rerio, Oncorhynchus mykiss, Bos taurus, Drosophila melanogaster, and Streptococcus suis BM407.

In some embodiments, the genetically modified yeast cell includes a heterologous nucleic acid encoding an enzyme that can catalyze the conversion of CMP-N-acetylneuraminate and lactose to 3′-siallyllactose and CMP, e.g., a CMP-N-acetylneuraminate-β-galactosamide-α-2,3-sialyltransferase. In some embodiments, the CMP-N-acetylneuraminate-β-galactosamide-α-2,3-sialyltransferase is from N. meningitides MC58. Other suitable CMP-N-acetylneuraminate-β-galactosamide-α-2,3-sialyltransferase sources include, for example and without limitation, Homo sapiens, Neisseria meningitidis alpha14, Pasteurella multocida subsp. multocida str. Pm70, Pasteurella multocida, and Rattus norvegicus.

In some embodiments, the genetically modified yeast cell is capable of producing 6′-sialyllactose. In addition to one or more heterologous nucleic acids encoding one or more of the aforementioned enzymes, the yeast can further include one or more heterologous nucleic acids encoding one or more of CMP-Neu5Ac synthetase, e.g., from Campylobacter jejuni, sialic acid synthase, e.g., from C. jejuni, UDP-N-acetylglucosamine 2-epimerase, e.g., from C. jejuni, UDP-N-acetylglucosamine-diphosphorylase, e.g., from E coli, and β-galactoside α-2,6-sialyltransferase, e.g., from Photobacterium sp. JT-ISH-224.

In some embodiments, the genetically modified yeast cell includes a heterologous nucleic acid encoding an enzyme that can catalyze the conversion of CMP-N-acetylneuraminate and lactose to 3′-sialyllactose and CMP, e.g., a β-galactoside-α-2,6-sialyltransferase. In some embodiments, the β-galactoside-α-2,6-sialyltransferase is from Photobacterium sp. JT-ISH-224. Other suitable β-galactoside-α-2,6-sialyltransferase sources include, for example and without limitation, Homo sapiens, Photobacterium damselae, Photobacterium leiognathi, and Photobacterium phosphoreum ANT-2200.

In some embodiments, the genetically modified yeast cell is Saccharomyces cerevisae. Saccharomyces cerevisae strains suitable for genetic modification and cultivation to produce HMOs as disclosed herein include, but are not limited to, Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, CEN.PK, CEN.PK2, and AL-1. In some embodiments, the host cell is a strain of Saccharomyces cerevisiae selected from the group consisting of PE-2, CAT-1, VR-1, BG-1, CR-1, and SA-1. In certain aspects, the strain of Saccharomyces cerevisiae is PE-2. In certain embodiments, the strain of Saccharomyces cerevisiae is CAT-1. In some aspects, the strain of Saccharomyces cerevisiae is BG-1.

In some embodiments, the genetically modified yeast cell is Saccharomyces cerevisiae, and in addition to heterologous nucleic acids encoding one or more of the aforementioned enzymes, the yeast can further include a heterologous nucleic acid encoding a lactose transporter. In some embodiments, the lactose transporter is a lactose permease, e.g., LAC12 from Kluyveromyces lactis. In some embodiments, the lactose permease is from Neurospora crassa, e.g., Cdt2. In some embodiments, the lactose permease is from Neofusicoccum parvum, e.g., Neofusicoccum parvum UCRNP2 (1287680). Other suitable lactose permease sources include, for example and without limitation, Scheffersomyces stipitis, Aspergillus lentulus, Emericella nidulans, Dacryopinax primogenitus, Microdochium bolleyi, Beauveria bassiana, Metarhizium robertsii, Phialocephala, Botryosphaeria parva, Moniliophthora roreri, Cordyceps fumosorosea, Diplodia seriata, Hypocrea jecorina, and Kluyveromyces marxianus.

In some embodiments, the genetically modified yeast cell is Kluyveromyces marxianus. Kluyveromyces marxianus can present several advantages for industrial production, including high temperature tolerance, acid tolerance, native uptake of lactose, and rapid growth rate. Beneficially, this yeast is genetically similar enough to Saccharomyces cerevisiae that similar or identical promoters and codon optimized genes can be used among the two yeast species. Furthermore, because Kluyveromyces marxianus has a native lactose permease, it is not necessary to introduce a heterologous nucleic acid to introduce this functionality. In some embodiments, at least a portion of the β-galactosidase gene (LAC4) required for metabolizing lactose is deleted in the genetically modified yeast. Thus, the modified Kluyveromyces marxianus strain is capable of importing lactose without consuming it. In some embodiments, the expression of the β-galactosidase gene in the genetically modified yeast is decreased relative to the expression in wild-type Kluyveromyces marxianus. Thus, the modified Kluyveromyces marxianus strain has reduced consumption of imported lactose.

In some embodiments, the genetically modified yeast cell includes a promoter that regulates the expression and/or stability of at least one of the one or more heterologous nucleic acids. In certain aspects, the promoter negatively regulates the expression and/or stability of the at least one heterologous nucleic acid. The promoter can be responsive to a small molecule that can be present in the culture medium of a fermentation of the modified yeast. In some embodiments, the small molecule is maltose or an analog or derivative thereof. In some embodiments, the small molecule is lysine or an analog or derivative thereof. Maltose and lysine can be attractive selections for the small molecule as they are relatively inexpensive, non-toxic, and stable.

In some embodiments, the promoter that regulates expression of the YOR1 variant ABC transporter polypeptide, e.g., the polypeptide of SEQ ID NOS: 2-15, or a variant thereof as described herein, is a relatively weak promoter, or an inducible promoter. Illustrative promoters include, for example, lower-strength GAL pathway promoters, such as GAL10, GAL2, and GAL3 promoters. Additional illustrative promoters for expressing an ABC transporter polypeptide include constitutive promoters from S. cerevisiae native promoters, such as the promoter from the native TDH3 gene. In some embodiments, a lower strength promoter provides a decrease in expression of at least 25%, or at least 30%, 40%, or 50%, or greater, when compared to a GAL1 promoter.

Expression of an YOR1 variant ABC transporter polypeptide, e.g., the polypeptide of SEQ ID NOS: 2-15, or a variant thereof as described herein can be accomplished by introducing into the host cells a nucleic acid comprising a nucleotide sequence encoding the ABC transporter polypeptide under the control of regulatory elements that permit expression in the host cell. In some embodiments, the nucleic acid is an extrachromosomal plasmid. In other embodiments, the nucleic acid is a chromosomal integration vector that can integrate the nucleotide sequence into the chromosome of the host cell.

In some embodiments, the one or more heterologous nucleic acids are introduced into the genetically modified yeast cells by using a gap repair molecular biology technique. In these methods, if the yeast has non-homologous end joining (NHEJ) activity, as is the case for Kluyveromyces marxianus, then the NHEJ activity in the yeast can be first disrupted in any of a number of ways. Further details related to genetic modification of yeast cells through gap repair can be found in U.S. Pat. No. 9,476,065, the full disclosure of which is incorporated by reference herein in its entirety for all purposes.

In some embodiments, the one or more heterologous nucleic acids are introduced into the genetically modified yeast cells by using one or more site-specific nucleases capable of causing breaks at designated regions within selected nucleic acid target sites. Examples of such nucleases include, but are not limited to, endonucleases, site-specific recombinases, transposases, topoisomerases, zinc finger nucleases, TAL-effector DNA binding domain-nuclease fusion proteins (TALENs), CRISPR/Cas-associated RNA-guided endonucleases, and meganucleases. Further details related to genetic modification of yeast cells through site specific nuclease activity can be found in U.S. Pat. No. 9,476,065, the full disclosure of which is incorporated by reference herein in its entirety for all purposes.

Described herein are specific genes and proteins useful in the methods, compositions, and organisms of the disclosure; however, it will be recognized that absolute identity to such genes is not necessary. For example, changes in a particular gene or polynucleotide comprising a sequence encoding a polypeptide or enzyme can be performed and screened for activity. Typically, such changes comprise conservative mutations and silent mutations. Such modified or mutated polynucleotides and polypeptides can be screened for expression of a functional enzyme using methods known in the art. Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given heterologous polypeptide of the disclosure. A native DNA sequence encoding the biosynthetic enzymes described above is referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties, e.g., charge or hydrophobicity. In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol. Biol. 25: 365-89).

Furthermore, any of the genes encoding the foregoing enzymes (or any others mentioned herein (or any of the regulatory elements that control or modulate expression thereof) can be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., Salmonella spp., or X dendrorhous.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art can be suitable to identify analogous genes and analogous enzymes. Techniques include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, e.g., as described herein or in Kiritani, K., Branched-Chain Amino Acids Methods Enzymology, 1970; then isolating the enzyme with said activity through purification; determining the protein sequence of the enzyme through techniques such as Edman degradation; design of PCR primers to the likely nucleic acid sequence; amplification of said DNA sequence through PCR; and cloning of said nucleic acid sequence. To identify homologous or similar genes and/or homologous or similar enzymes, suitable techniques also include comparison of data concerning a candidate gene or enzyme with databases such as BRENDA, KEGG, or MetaCYC. The candidate gene or enzyme can be identified within the above mentioned databases in accordance with the teachings herein.

### Methods of Producing Human Milk Oligosaccharides

Also provided herein are methods of producing one or more HMOs. The methods include providing a population of genetically modified yeast cells capable of producing one or more HMOs, which genetically modified yeast cells are also genetically modified to express an ABC transporter polypeptide, e.g., the polypeptide of SEQ ID NOS: 2-15, or a variant thereof as described herein as described herein. Each yeast cell of the population can include one more heterologous nucleic acids that encode the ABC transporter polypeptide and an enzyme of a HMO biosynthetic pathway. In some embodiments, the population includes any of the yeast cells as disclosed herein and discussed above. The methods further include providing a culture medium and culturing the yeast cells in the culture medium under conditions suitable for the yeast cells to produce the one or more milk oligosaccharides.

The culturing can be performed in a suitable culture medium in a suitable container, including but not limited to a cell culture plate, a flask, or a fermentor. Any suitable fermentor may be used, including, but not limited to, a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric et al., in Ullmann's Encyclopedia of Industrial Chemistry, Sixth Edition, Volume 12, pages 398-473, Wiley-VCH Verlag GmbH & Co. KDaA, Weinheim, Germany. Further, the methods can be performed at any scale of fermentation known in the art to support industrial production of microbial products. Materials and methods for the maintenance and growth of cell cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

In some embodiments, the culturing is carried out for a period of time sufficient for the transformed population to undergo a plurality of doublings until a desired cell density is reached. In some embodiments, the culturing is carried out for a period of time sufficient for the host cell population to reach a cell density (OD600) of between 0.01 and 400 in the fermentation vessel or container in which the culturing is being carried out. The culturing can be carried out until the cell density is, for example, between 0.1 and 14, between 0.22 and 33, between 0.53 and 76, between 1.2 and 170, or between 2.8 and 400. In terms of upper limits, the culturing can be carried until the cell density is no more than 400, e.g., no more than 170, no more than 76, no more than 33, no more than 14, no more than 6.3, no more than 2.8, no more than 1.2, no more than 0.53, or no more than 0.23. In terms of lower limits, the culturing can be carried out until the cell density is greater than 0.1, e.g., greater than 0.23, greater than 0.53, greater than 1.2, greater than 2.8, greater than 6.3, greater than 14, greater than 33, greater than 76, or greater than 170. Higher cell densities, e.g., greater than 400, and lower cell densities, e.g., less than 0.1, are also contemplated.

In other embodiments, the culturing is carried for a period of time, for example, between 12 hours and 92 hours, e.g., between 12 hours and 60 hours, between 20 hours and 68 hours, between 28 hours and 76 hours, between 36 hours and 84 hours, or between 44 hours and 92 hours. In some embodiments, the culturing is carried out for a period of time, for example, between 5 days and 20 days, e.g., between 5 days and 14 days, between 6.5 days and 15.5 days, between 8 days and 17 days, between 9.5 days and 18.5 days, or between 11 days and 20 days. In terms of upper limits, the culturing can be carried out for less than 20 days, e.g., less than 18.5 days, less than 17 days, less than 15.5 days, less than 14 days, less than 12.5 day, less than 11 days, less than 9.5 days, less than 8 days, less than 6.5 days, less than 5 day, less than 92 hours, less than 84 hours, less than 76 hours, less than 68 hours, less than 60 hours, less than 52 hours, less than 44 hours, less than 36 hours, less than 28 hours, or less than 20 hours. In terms of lower limits, the culturing can be carries out for greater than 12 hours, e.g., greater than 20 hours, greater than 28 hours, greater than 36 hours, greater than 44 hours, greater than 52 hours, greater than 60 hours, greater than 68 hours, greater than 76 hours, greater than 84 hours, greater than 92 hours, greater than 5 days, greater than 6.5 days, greater than 8 days, greater than 9.5 days, greater than 11 days, greater than 12.5 days, greater than 14 days, greater than 15.5 days, greater than 17 days, or greater than 18.5 days. Longer culturing times, e.g., greater than 20 days, and shorter culturing times, e.g., less than 5 hours, are also contemplated.

In certain embodiments, the production of the one or more HMOs by the population of genetically modified yeast is inducible by an inducing compound. Such yeast can be manipulated with ease in the absence of the inducing compound. The inducing compound is then added to induce the production of the HMOs by the yeast. In other embodiments, production of the one or more HMOs by the yeast is inducible by changing culture conditions, such as, for example, the growth temperature, media constituents, and the like.

In certain embodiments, an inducing agent is added during a production stage to activate a promoter or to relieve repression of a transcriptional regulator associated with a biosynthetic pathway to promote production of HMOs. In certain embodiments, an inducing agent is added during a build stage to repress a promoter or to activate a transcriptional regulator associated with a biosynthetic pathway to repress the production of HMOs, and an inducing agent is removed during the production stage to activate a promoter to relieve repression of a transcriptional regulator to promote the production of HMOs. The term “genetic switch” is used herein to refer to the use of a promoter or other genetic elements to control activation or de-activation of the biosynthetic pathway for the one or more HMOs. Illustrative examples of useful inducing agents or genetic switches are described in, e.g., PCT Application Publications WO2015/020649, WO2016/210343, and WO2016210350, which are incorporated herein by reference in their entirety.

As discussed above, in some embodiments, the provided genetically modified yeast cell includes a promoter that regulates the expression and/or stability of at least one of the one or more heterologous nucleic acids. Thus, in certain embodiments, the promoter can be used to control the timing of gene expression and/or stability of proteins, for example, an ABC transporter polypeptide, e.g., the polypeptide of SEQ ID NOS: 2-15, or a variant thereof as described herein, or enzymes of a biosynthetic pathway for producing HMOs in genetically modified yeast cells during fermentation.

In some embodiments, when fermentation of a genetically modified yeast cell is carried out in the presence of a small molecule, e.g., at least about 0.1% maltose or lysine, HMO production is substantially reduced or turned off. When the amount of the small molecule in the fermentation culture medium is reduced or eliminated, HMO production is turned on or increased. Such a system enables the use of the presence or concentration of a selected small molecule in a fermentation medium as a switch for the production of non-catabolic, e.g., HMO, compounds. Controlling the timing of non-catabolic compound production to occur only when production is desired redirects the carbon flux during the non-production phase into cell maintenance and biomass. This more efficient use of carbon can greatly reduce the metabolic burden on the host cells, improve cell growth, increase the stability of the heterologous genes, reduce strain degeneration, and/or contribute to better overall health and viability of the cells.

In some embodiments, the fermentation method comprises a two-step process that utilizes a small molecule as a switch to affect the “off” and “on” stages. In the first step, i.e., the “build” stage, step (a) wherein production of the compound is not desired, the genetically modified yeast are grown in a growth or “build” medium comprising the small molecule in an amount sufficient to induce the expression of genes under the control of a responsive promoter, and the induced gene products act to negatively regulate production of the non-catabolic compound. After transcription of the fusion DNA construct under the control of a maltose-responsive or lysine-responsive promoter, the stability of the fusion proteins is post-translationally controlled. In the second step, i.e., the “production” stage, step (b), the fermentation is carried out in a culture medium comprising a carbon source wherein the small molecule is absent or in sufficiently low amounts such that the activity of a responsive promoter is reduced or inactive and the fusion proteins are destabilized. As a result, the production of the heterologous non-catabolic compound by the host cells is turned on or increased.

In other embodiments, a responsive promoter can be operably linked to one or more heterologous nucleic acids encoding one or more enzymes of a HMO pathway. The presence of an activating amount of the small molecule in the culture medium increases the expression of the one or more enzymes of the biosynthetic pathway. In these embodiments, the presence of a sufficient amount of maltose or lysine in the culture medium will increase expression of one or more enzymes of the biosynthetic pathway, and the fusion enzymes are stabilized in the presence of the small molecule.

In some embodiments, the culture medium is any culture medium in which a genetically modified yeast capable of producing an HMO can subsist, i.e., maintain growth and viability. In some embodiments, the culture medium is an aqueous medium comprising assimilable carbon, nitrogen, and phosphate sources. Such a medium can also include appropriate salts, minerals, metals, and other nutrients. In some embodiments, the carbon source and each of the essential cell nutrients, are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

In another embodiment, the method of producing HMOs comprises culturing host cells in separate build and production culture media. For example, the method can comprise culturing the genetically modified host cell in a build stage wherein the cell is cultured under non-producing conditions, e.g., non-inducing conditions, to produce an inoculum, then transferring the inoculum into a second fermentation medium under conditions suitable to induce HMO production, e.g., inducing conditions, and maintaining steady state conditions in the second fermentation stage to produce a cell culture containing HMOs.

In some embodiments, the culture medium comprises sucrose and lactose. In some embodiments, the carbon sources in the culture medium consist essentially of sucrose and lactose. In some embodiments, the carbon sources in the culture medium consist of sucrose and lactose. In some embodiments, the mass ratio of the sucrose to the lactose is selected to influence, adjust, or control the relative production rates of HMOs produced by the yeast cells. Controlling the composition of the produced HMOs in this way can advantageously permit the increasing of desired products, the decreasing of undesired products, the targeting of a desired product ratio, and the simplification of downstream product separation processes.

The mass ratio of the sucrose to the lactose in the culture medium can be, for example, between 4 and 40, e.g., between 4 and 25.6, between 7.6 and 29.2, between 11.2 and 32.8, between 14.8 and 36.4, or between 18.4 and 40. In terms of upper limits, the mass ratio of the sucrose to the lactose can be less than 40, e.g., less than 36.4, less than 32.8, less than 29.2, less than 25.6, less than 22, less than 18.4, less than 14.8, less than 11.2, or less than 7.6. In terms of lower limits, the mass ratio of the sucrose to the lactose can be greater than 4, e.g., greater than 7.6, greater than 11.2, greater than 14.8, greater than 18.4, greater than 22, greater than 25.6, greater than 29.2, greater than 32.8, or greater than 36.4. Higher ratios, e.g., greater than 40, and lower ratios, e.g., less than 4, are also contemplated.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. In some embodiments, the addition of a nitrogen source to the culture medium beyond a certain concentration is not advantageous for the growth of the yeast. As a result, the concentration of the nitrogen sources, in the culture medium can be less than about 20 g/L, e.g., less than about 10 g/L or less than about 5 g/L. Further, in some instances it may be desirable to allow the culture medium to become depleted of the nitrogen sources during culturing.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, e.g., greater than about 2.0 g/L or greater than about 5.0 g/L. In some embodiments, the addition of phosphate to the culture medium beyond certain concentrations is not advantageous for the growth of the yeast. Accordingly, the concentration of phosphate in the culture medium can be less than about 20 g/L, e.g., less than about 15 g/L or less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, e.g., greater than about 1.0 g/L or greater than about 2.0 g/L. In some embodiments, the addition of magnesium to the culture medium beyond certain concentrations is not advantageous for the growth of the yeast. Accordingly, the concentration of magnesium in the culture medium can be less than about 10 g/L, e.g, less than about 5 g/L or less than about 3 g/L. Further, in some instances it may be desirable to allow the culture medium to become depleted of a magnesium source during culturing.

In some embodiments, the culture medium can also include a biologically acceptable chelating agent, such as the dihydrate of trisodium citrate. In such instance, the concentration of a chelating agent in the culture medium can be greater than about 0.2 g/L, e.g., greater than about 0.5 g/L or greater than about 1 g/L. In some embodiments, the addition of a chelating agent to the culture medium beyond certain concentrations is not advantageous for the growth of the yeast. Accordingly, the concentration of a chelating agent in the culture medium can be less than about 10 g/L, e.g., less than about 5 g/L or less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, e.g., within the range of from about 20 mg/L to about 1000 mg/L or in the range of from about 50 mg/L to about 500 mg/L.

The culture medium can also include sodium chloride. Typically, the concentration of sodium chloride in the culture medium is within the range of from about 0.1 g/L to about 5 g/L, e.g., within the range of from about 1 g/L to about 4 g/L or in the range of from about 2 g/L to about 4 g/L.

In some embodiments, the culture medium can also include trace metals. Such trace metals can be added to the culture medium as a stock solution that, for convenience, can be prepared separately from the rest of the culture medium Typically, the amount of such a trace metals solution added to the culture medium is greater than about 1 ml/L, e.g., greater than about 5 mL/L, and more preferably greater than about 10 mL/L. In some embodiments, the addition of a trace metals to the culture medium beyond certain concentrations is not advantageous for the growth of the yeast. Accordingly, the amount of such a trace metals solution added to the culture medium can be less than about 100 mL/L, e.g., less than about 50 mL/L or less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, inositol, pyridoxine-HCl, thiamine-HCl, and combinations thereof. Such vitamins can be added to the culture medium as a stock solution that, for convenience, can be prepared separately from the rest of the culture medium In some embodiments, the addition of vitamins to the culture medium beyond certain concentrations is not advantageous for the growth of the yeast.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, e.g., during the production stage of the fermentation. In some embodiments, the culture may be supplemented with relatively high concentrations of such components at the outset, for example, of the production stage, so that growth and/or HMO production is supported for a period of time before additions are required. The preferred ranges of these components can be maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those of ordinary skill in the art, the rate of consumption of nutrient increases during culture as the cell density of the medium increases. Moreover, to avoid introduction of foreign microorganisms into the culture medium, addition can be performed using aseptic addition methods, as are known in the art. In addition, a small amount of anti-foaming agent may be added during the culture.

The temperature of the culture medium can be any temperature suitable for growth of the genetically modified yeast population and/or production of the one or more HMOs. For example, prior to inoculation of the culture medium with an inoculum, the culture medium can be brought to and maintained at a temperature in the range of from about 20° C. to about 45° C., e.g., to a temperature in the range of from about 25° C. to about 40° C. or of from about 28° C. to about 32° C. For example, the culture medium can be brought to and maintained at a temperature of 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or 40° C.

The pH of the culture medium can be controlled by the addition of acid or base to the culture medium In such cases when ammonia is used to control pH, it also conveniently serves as a nitrogen source in the culture medium In some embodiments, the pH is maintained from about 3.0 to about 8.0, e.g., from about 3.5 to about 7.0 or from about 4.0 to about 6.5.

In some embodiments, the genetically modified yeast cells produce 2′-fucosyllactose. The concentration of produced 2′-fucosyllactose in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 2′-fucosyllactose in the culture medium can be, for example, between 5 g/l and 100 g/l, e.g., between 5 g/l and 50 to 90 g/l, between 10 g/l and 80 g/l, between 10 g/l and 75 g/l, between 20 g/l and 80 g/l, or between 20 g/l and 80 g/l. In some embodiments, the 2′-fucosyllactose concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 2′-fucosyllactose can be 40 g/l or greater, e.g., 50 g/l, 60 g/l 70 g/l 80 g/l, 90 g/l e.g., or greater. For example, in some embodiments, concentrations of produced 2′-fucosyllactose in the culture medium can be 100 g/l or greater. In some embodiments, expression of a variant YOR1 polypeptide, e.g., the polypeptide of SEQ ID NOS: 2-15, or a variant thereof as described herein, enhances production of 2′-fucosyllactose, compared to a counterpart control strain that is not modified to express the ABC transporter polypeptide, is enhanced by at least 5%, or at least 10%, 20%, 30%, 40%, 50%, 60%, 70%, 80%, 90%, 100%, or greater, compared to the control.

The yield of produced 2′-fucosyllactose on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 2′-fucosyllactose on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g or 0.26 g/g, or greater. In some embodiments, expression of an ABC transporter polypeptide, e.g., the polypeptide of SEQ ID NOS: 2-15, or a variant thereof as described herein, enhances production of 2′-fucosyllactose, compared to a counterpart control strain that is not modified to express the ABC transporter polypeptide, by at least 5%, or at least 10%, 20%, 30%, 40%, 50%, 60%, 70%, 80%, 90%, 100%, or greater, compared to the control.

In some embodiments, the genetically modified yeast cells produce difucosyllactose. The concentration of produced difucosyllactose in the culture medium can be, for example, between 5 g/l and 40 g/l, e.g., between 5 g/l and 26 g/l, between 8.5 g/l and 29.5 g/l, between 12 g/l and 33 g/l, between 15.5 g/l and 36.5 g/l, or between 19 g/l and 40 g/l. In terms of upper limits, the 2′-fucosyllactose concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. Higher concentrations, e.g., greater than 40 g/l, are also contemplated.

In some embodiments, it is desirable to minimize the amount of difucosyllactose produced by the genetically modified yeast cells relative the amount of 2′-fucosyllactose produced. The mass of difucosyllactose produced by the yeast cells per g of 2′-fucosyllactose produced by the yeast cells can be, for example, between 0.001 g and 5 g e.g, between 0.01 g and 5 g, between 0.1 g and 5 g, between 0.2 g and 4.2 g, between 0.2 g and 2.6 g, between 0.6 g and 3 g, between 1 g and 3.4 g, between 1.4 g and 3.8 g, or between 1.8 g and 4.2 g. In terms of upper limits, the mass of difucosyllactose produced per g of 2′-fucosyllactose can be less than 4.2 g, e.g., less than 3.8 g, less than 3.4 g, less than 3 g, less than 2.6 g, less than 2.2 g, less than 1.8 g, less than 1.4 g, less than 1 g, less than 0.6 g, or less than 0.2 g. In terms of lower limits, the mass of difucosyllactose produced per g of 2′-fucosyllactose can be greater than 0.2 g, e.g., greater than 0.6 g, greater than 1 g, greater than 1.4 g, greater than 1.8 g, greater than 2.2 g, greater than 2.6 g, greater than 3 g, greater than 3.4 g, or greater than 3.8 g. Higher mass ratios, e.g., greater than 4.2 g/g, and lower mass ratios, e.g., less than 0.2 g/g, are also contemplated.

In some embodiments, the genetically modified yeast cells produce lacto-N-tetraose. The concentration of produced lacto-N-tetraose in the culture medium can be, for example, between 0.5 g/l and 8 g/l, e.g., between 0.5 g/l and 2.6 g/l, between 0.7 g/l and 3.5 g/l, between 0.9 g/l and 4.6 g/l, between 1.1 g/l and 6.1 g/l, or between 1.5 g/l and 8 g/l. In terms of upper limits, the lacto-N-tetraose concentration can be greater than 0.5 g/l, e.g., greater than 0.7 g/l, greater than 0.9 g/l, greater than 1.1 g/l, greater than 1.5 g/l, greater than 2 g/l, greater than 2.6 g/l, greater than 3.5 g/l, greater than 4.6 g/l, or greater than 6 g/l. Higher concentrations, e.g., greater than 8 g/l, are also contemplated. In some embodiments, expression of a variant YOR1 polypeptide, e.g., the polypeptide of SEQ ID NOS: 2-15, or a variant thereof as described herein, enhances production of lacto-N-tetraose, compared to a counterpart control strain that is not modified to express the ABC transporter polypeptide, by at least 5%, or at least 10%, 20%, 30%, 40%, 50%, 60%, 70%, 80%, 90%, 100%, or greater, compared to the control.

In some embodiments, the genetically modified yeast cells produce lacto-N-neotetraose. The concentration of produced lacto-N-neotetraose in the culture medium can be, for example, between 0.5 g/l and 30 g/l, e.g., between 0.5 g/l and 5.8 g/l, between 0.8 g/l and 8.8 g/l, between 1.1 g/l and 13 g/l, between 1.7 g/l and 20 g/l, or between 2.6 g/l and 30 g/l. In terms of upper limits, the lacto-N-neotetraose concentration can be greater than 0.5 g/l, e.g., greater than 0.8 g/l, greater than 1.1 g/l, greater than 1.7 g/l, greater than 2.6 g/l, greater than 3.9 g/l, greater than 5.8 g/l, greater than 8.8 g/l, greater than 13 g/l, or greater than 20 g/l. Higher concentrations, e.g., greater than 30 g/l, are also contemplated. In some embodiments, expression of a variant YOR1 polypeptide, e.g., the polypeptide of SEQ ID NOS: 2-15, or a variant thereof as described herein, enhances production of lacto-N-neotetraose, compared to a counterpart control strain that is not modified to express the variant YOR1 polypeptide, by at least 5%, or at least 10%, 20%, 30%, 40%, 50%, 60%, 70%, 80%, 90%, 100%, or greater, compared to the control.

In some embodiments, the genetically modified yeast cells produce 3-fucosyllactose. The concentration of produced 3-fucosyllactose in the culture medium can be, for example, between 0.05 g/l and 3 g/l, e.g., between 0.05 g/l and 2 g/l, between 0.07 g/l and 0.35 g/l, between 0.09 g/l and 0.46 g/l, between 0.11 g/l and 0.61 g/l, or between 0.15 g/l and 0.8 g/l. In terms of upper limits, the 3-fucosyllactose concentration can be greater than 0.05 g/l, e.g., greater than 0.07 g/l, greater than 0.09 g/l, greater than 0.11 g/l, greater than 0.15 g/l, greater than 0.2 g/l, greater than 0.26 g/l, greater than 0.35 g/l, greater than 0.46 g/l, or greater than 0.6 g/l. Higher concentrations, e.g., greater than 0.8 g/l, are also contemplated. In some embodiments, expression of a variant YOR1 polypeptide, e.g., the polypeptide of SEQ ID NOS: 2-7, or a variant thereof as described herein, enhances production of 3-fucosyllactose, compared to a counterpart control strain that is not modified to express the variant YOR1 polypeptide, by at least 5%, or at least 10%, 20%, 30%, 40%, 50%, 60%, 70%, 80%, 90%, 100%, or greater, compared to the control.

In some embodiments, the genetically modified yeast cells produce 3′-sialyllactose. The concentration of produced 3′-sialyllactose in the culture medium can be, for example, between 0.1 g/l and 1.6 g/l, e.g., between 0.1 g/l and 0.53 g/l, between 0.13 g/l and 0.7 g/l, between 0.17 g/l and 0.92 g/l, between 0.23 g/l and 1.2 g/l, or between 0.3 g/l and 1.6 g/l. In terms of upper limits, the 3′-sialyllactose concentration can be greater than 0.1 g/l, e.g., greater than 0.13 g/l, greater than 0.17 g/l, greater than 0.23 g/l, greater than 0.3 g/l, greater than 0.4 g/l, greater than 0.53 g/l, greater than 0.7 g/l, greater than 0.92 g/l, or greater than 1.2 g/l. Higher concentrations, e.g., greater than 1.6 g/l, are also contemplated. In some embodiments, expression of a variant YOR1 polypeptide, e.g., the polypeptide of SEQ ID NOS: 2-15, or a variant thereof as described herein, enhances production of 3′-sialyllactose, compared to a counterpart control strain that is not modified to express the ABC transporter polypeptide, by at least 5%, or at least 10%, 20%, 30%, 40%, 50%, 60%, 70%, 80%, 90%, 100%, or greater, compared to the control.

In some embodiments, the genetically modified yeast cells produce 6′-sialyllactose. The concentration of produced 6′-sialyllactose in the culture medium can be, for example, between 0.25 g/l and 20 g/l, e.g., between 0.25 g/l and 15 g/l, between 0.33 g/l and 20 g/l, between 0.44 g/l and 20 g/l, between 0.57 g/l and 20 g/l, or between 0.76 g/l and 20 g/l. In terms of upper limits, the 3′-sialyllactose concentration can be greater than 20 g/l, e.g., or greater than 10 g/l. Higher concentrations, e.g., greater than 20 g/l, are also contemplated. In some embodiments, expression of a variant YOR1 polypeptide, e.g., the polypeptide of SEQ ID NOS: 2-15, or a variant thereof as described herein, enhances production of 6′-sialyllactose, compared to a counterpart control strain that is not modified to express the ABC transporter polypeptide, by at least 5%, or at least 10%, 20%, 30%, 40%, 50%, 60%, 70%, 80%, 90%, 100%, or greater, compared to the control.

### Fermentation Compositions

Also provided are fermentation compositions including a population of genetically modified yeast cells. The yeast cells can include any of the yeast cells disclosed herein and discussed above. In some embodiments, the fermentation composition further includes at least one HMO produced from the yeast cells. The at least one HMO in the fermentation composition can include, for example, 2′-fucosyllactose, difucosyllactose, 3-fucosyllactose, lacto-N-tetraose, lacto-N-neotetraose, 3′-sialyllactose, or 6′-sialyllactose. In some embodiments, the fermentation composition includes at least two HMOs. The at least two HMOs in the fermentation composition can include, for example, 2′-fucosyllactose and difucosyllactose, 2′-fucosyllactose and 3-fucosyllactose, 2′-fucosyllactose and lacto-N-tetraose, 2′-fucosyllactose and lacto-N-neotetraose, 2′-fucosyllactose and 3′-sialyllactose, 2′-fucosyllactose and 6′-sialyllactose, difucosyllactose and 3-fucosyllactose, difucosyllactose and lacto-N-tetraose, difucosyllactose and lacto-N-neotetraose, difucosyllactose and 3′-sialyllactose, difucosyllactose and 6′-sialyllactose, 3-fucosyllactose and lacto-N-tetraose, 3-fucosyllactose and lacto-N-neotetraose, 3-fucosyllactose and 3′-sialyllactose, 3-fucosyllactose and 6′-sialyllactose, lacto-N-tetraose and lacto-N-neotetraose, lacto-N-tetraose and 3′-sialyllactose, lacto-N-tetraose and 6′-sialyllactose, lacto-N-neotetraose and 3′-sialyllactose, lacto-N-neotetraose and 6′-sialyllactose, or 3′-sialyllactose and 6′-sialyllactose.

In some embodiments, the fermentation composition includes at least three HMOs produced from the yeast cells. The at least three HMOs in the fermentation composition can include, for example, 2′-fucosyllactose, difucosyllactose, and 3-fucosyllactose; 2′-fucosyllactose, difucosyllactose, and lacto-N-tetraose; 2′-fucosyllactose, difucosyllactose, and lacto-N-neotetraose; 2′-fucosyllactose, difucosyllactose, and 3′-sialyllactose; 2′-fucosyllactose, difucosyllactose, and 6′-sialyllactose; 2′-fucosyllactose, 3-fucosyllactose, and lacto-N-tetraose; 2′-fucosyllactose, 3-fucosyllactose, and lacto-N-neotetraose; 2′-fucosyllactose, 3-fucosyllactose, and 3′-sialyllactose; 2′-fucosyllactose, 3-fucosyllactose, and 6′-sialyllactose; 2′-fucosyllactose, lacto-N-tetraose, and lacto-N-neotetraose; 2′-fucosyllactose, lacto-N-tetraose, and 3′-sialyllactose; 2′-fucosyllactose, lacto-N-tetraose, and 6′-sialyllactose; 2′-fucosyllactose, lacto-N-neotetraose, and 3′-sialyllactose; 2′-fucosyllactose, lacto-N-neotetraose, and 6′-sialyllactose; 2′-fucosyllactose, 3′-sialyllactose, and 6′-sialyllactose; difucosyllactose, 3-fucosyllactose, and lacto-N-tetraose; difucosyllactose, 3-fucosyllactose, and lacto-N-neotetraose; difucosyllactose, 3-fucosyllactose, and 3′-sialyllactose; difucosyllactose, 3-fucosyllactose, and 6′-sialyllactose; difucosyllactose, lacto-N-tetraose, and lacto-N-neotetraose; difucosyllactose, lacto-N-tetraose, and 3′-sialyllactose; difucosyllactose, lacto-N-tetraose, and 6′-sialyllactose; difucosyllactose, lacto-N-neotetraose, and 3′-sialyllactose; difucosyllactose, lacto-N-neotetraose, and 6′-sialyllactose; difucosyllactose, 3′-sialyllactose, and 6′-sialyllactose; 3-fucosyllactose, lacto-N-tetraose, and lacto-N-neotetraose; 3-fucosyllactose, lacto-N-tetraose, and 3′-sialyllactose; 3-fucosyllactose, lacto-N-tetraose, and 6′-sialyllactose; 3-fucosyllactose, lacto-N-neotetraose, and 3′-sialyllactose; 3-fucosyllactose, lacto-N-neotetraose, and 6′-sialyllactose; 3-fucosyllactose, 3′-sialyllactose, and 6′-sialyllactose; lacto-N-tetraose, lacto-N-neotetraose, and 3′-sialyllactose; lacto-N-tetraose, lacto-N-neotetraose, and 6′-sialyllactose; or lacto-N-neotetraose, 3′-sialyllactose, and 6′-sialyllactose. In some embodiments, the fermentation composition includes at least four HMOs produced from the yeast cells. In some embodiments, the fermentation composition includes at least five HMOs produced from the yeast cells. In some embodiments, the fermentation composition includes at least six HMOs produced from the yeast cells. In some embodiments, the fermentation composition includes at least seven HMOs produced from the yeast cells.

The mass fraction of difucosyllactose within the one or more produced HMOs can be, for example, between 0 and 50%, e.g., between 0 and 30%, between 5% and 35%, between 10% and 40%, between 15% and 45%, or between 20% and 40%. In terms of upper limits, the mass fraction of difucosyllactose in the HMOs can be less than 50%, e.g., less than 45%, less than 40%, less than 35%, less than 30%, less than 25%, less than 20%, less than 15%, less than 10%, or less than 5%.

### Methods of Recovering Human Milk Oligosaccharides

Also provided are methods of recovering one or more HMOs from a fermentation composition. In some embodiments, the fermentation composition is any of the fermentation composition disclosed herein and described above. The method includes separating at least a portion of a population of yeast cells from a culture medium. In some embodiments, the separating includes centrifugation. In some embodiments, the separating includes filtration.

While some portion of the one or more HMOs produced by the cells during fermentation can be expected to partition with the culture medium during the separation of the yeast cells from the medium, some of the HMOs can be expected to remain associated with the yeast cells. One approach to capturing this cell-associated product and improving overall recovery yields is to rinse the separated cells with a wash solution that is then collected. It has now been found that the effectiveness of such a rinse can be significantly increased by heating the wash solution prior to its use.

Accordingly, the provided recovery methods further include contacting the separated yeast cells with a heated wash liquid. In some embodiments, the heated wash liquid is a heated aqueous wash liquid. In some embodiments, the heated wash liquid consists of water. In some embodiments, the heated wash liquid includes one or more other liquid or dissolved solid components.

The temperature of the heated aqueous wash liquid can be, for example, between 30° C. and 90° C., e.g., between 30° C. and 66° C., between 36° C. and 72° C., between 42° C. and 78° C., between 48° C. and 84° C., or between 54° C. and 90° C. In terms of upper limits, the wash temperature can be less than 90° C., e.g., less than 84° C., less than 78° C., less than 72° C., less than 66° C., less than 60° C., less than 54° C., less than 48° C., less than 42° C., or less than 36° C. In terms of lower limits, the wash temperature can be greater than 30° C., e.g., greater than 36° C., greater than 42° C., greater than 48° C., greater than 54° C., greater than 60° C., greater than 66° C., greater than 72° C., greater than 78° C., or greater than 84° C. Higher temperatures, e.g., greater than 90° C., and lower temperatures, e.g., less than 30° C., are also contemplated.

The method further includes, subsequent to the contacting of the separated yeast cells with the heated wash liquid, removing the wash liquid from the yeast cells. In some embodiments, the removed wash liquid is combined with the separated culture medium and further processed to isolate the produced one or more HMOs. In some embodiments, the removed wash liquid and the separated culture medium are further processed independently of one another. In some embodiments, the removal of the wash liquid from the yeast cells includes centrifugation. In some embodiments, the removal of the wash liquid from the yeast cells includes filtration.

The recovery yield can be such that, for at least one of the one or HMOs produced from the yeast cells, the mass fraction of the produced at least one HMO recovered in the combined culture medium and wash liquid is, for example, between 70% and 100%, e.g., between 70% and 88%, between 73% and 91%, between 76% and 94%, between 79% and 97%, or between 82% and 100%. In terms of lower limits, the recovery yield of at least one of the one or more HMOs can be greater than 70%, e.g., greater than 73%, greater than 76%, greater than 79%, greater than 82%, greater than 85%, greater than 88%, greater than 91%, greater than 94%, or greater than 97%. The recovery yield can be such that, for each of the one or more HMOs produced from the yeast cells, the mass fraction recovered in the combined culture medium and wash liquid is, for example, between 70% and 100%, e.g., between 70% and 88%, between 73% and 91%, between 76% and 94%, between 79% and 97%, or between 82% and 100%. In terms of lower limits, the recovery yield of each of the one or more HMOs can be greater than 70%, e.g., greater than 73%, greater than 76%, greater than 79%, greater than 82%, greater than 85%, greater than 88%, greater than 91%, greater than 94%, or greater than 97%.

While the compositions and methods provided herein have been described with respect to a limited number of embodiments, one or more features from any of the embodiments described herein or in the figures can be combined with one or more features of any other embodiment described herein in the figures without departing from the scope of the disclosure. No single embodiment is representative of all aspects of the methods or compositions. In certain embodiments, the methods can include numerous steps not mentioned herein. In certain embodiments, the methods do not include any steps not enumerated herein. Variations and modifications from the described embodiments exist.

### Methods of Treating a Fermentation Composition

Also provided are methods of treating a fermentation composition. The treatment methods are particularly useful for increasing the yield of 2′-fucosyllactose within fermentation compositions that include difucosyllactose. In some embodiments, the fermentation composition is any of the fermentation composition disclosed herein and described above. The method includes providing a fermentation composition comprising difucosyllactose. The concentration of difucosyllactose in the fermentation composition can be as described above. The method further includes contacting the fermentation with an enzyme capable of converting difucosyllactose to 2′-fucosyllactose, e.g., an α1-3,4 fucosidase. The α1-3,4 fucosidase can be encoded by a gene engineered into a strain of the fermentation, such that the α1-3,4 fucosidase is expressed during the fermentation. The α1-3,4 fucosidase can be exogenously added to the fermentation composition as part of a downstream processing protocol. Suitable α1-3,4 fucosidase sources include, for example and without limitation, Bacteroides thetaiotaomicron, Bifidobacterium bifidum, Bifidobacterium longum, Bifidobacterium longum subsp. infantis, Clostridium perfringens, Lactobacillus casei, Paenibacillus thiaminolyticus, Pseudomonas putida, Thermotoga maritima, Xanthomonas campestris pv. campestris, Arabidopsis thaliana, and Rattus norvegicus.

The contacting of the fermentation composition with the α1-3,4 fucosidase is under conditions suitable for converting at least a portion of the difucosyllactose to 2′-fucosyllactose. The percentage of initial difucosyllactose converted by the α1-3,4 fucosidase can be, for example, between 20% and 100%, e.g., between 20% and 68%, between 28% and 76%, between 36% and 84%, between 44% and 92%, or between 52% and 100%. In terms of lower limits, the percent conversion of the difucosyllactose can be greater than 20%, e.g., greater than 28%, greater than 36%, greater than 44%, greater than 52%, greater than 60%, greater than 68%, greater than 76%, greater than 84%, or greater than 92%. In some embodiments, the fermentation composition further comprises 3-fucosyllactose, and the contacting of the fermentation composition with the α1-3,4 fucosidase also includes reducing the level of 3-fucosyllactose in the fermentation composition, further improving 2′-fucosyllactose purity in the composition.

All publications and patent applications mentioned in this specification are herein incorporated by reference to the same extent as if each individual publication or patent application was specifically and individually indicated to be incorporated by reference. Although the claimed subject matter has been described in some detail by way of illustration and example for purposes of clarity of understanding, it will be readily apparent to those of ordinary skill in the art in light of the teachings herein that certain changes and modifications may be made thereto without departing from the spirit or scope of the appended claims.

### EXAMPLES

The present disclosure will be better understood in view of the following non-limiting examples. The following examples are intended for illustrative purposes only and do not limit in any way the scope of the present invention.

### Example 1. YOR1 Site Saturation Mutagenesis (SSM) Library for Improved Export of 2′-FL

In order to identify variant YOR1 polypeptides with improved 2′-FL export a site saturation mutagenesis (SSM) library was prepared and screened for various activities indicative of improved export of 2′-FL. The SSM library targeted 420 amino acid residues in the YOR1 polypeptide that were predicted to have the highest likelihood of impacting transmembrane transport activity. Individual mutants were screened for: 1) increased 2′-FL titer and increased 2′-FL/DFL ratio; 2) increased 2′-FL titer and lowest lactose titer remaining; and 3) highest increase in total FL equivalents.

The rationale for screening for increased 2′-FL titer and increased 2′-FL/DFL ratio is that enhanced 2′-FL export would lead to less intracellular 2′-FL available for the production of the DFL off-product. The rationale for screening for increased 2′-FL titer and lowest lactose titer remaining is that these mutants have the highest consumption of the lactose substrate which would be indicative of increased activity of fucosyltransferase due to improved 2′-FL export. The rationale for screening for an increase in total FL equivalents is that even though they may not show an improved 2′-FL to DFL ratio, it is possible that these mutants generate the most flux due to the export of both 2′-FL and DFL. The increase export of DFL would not be problematic in a commercial 2′-FL because the production strains are unlikely to make DFL.

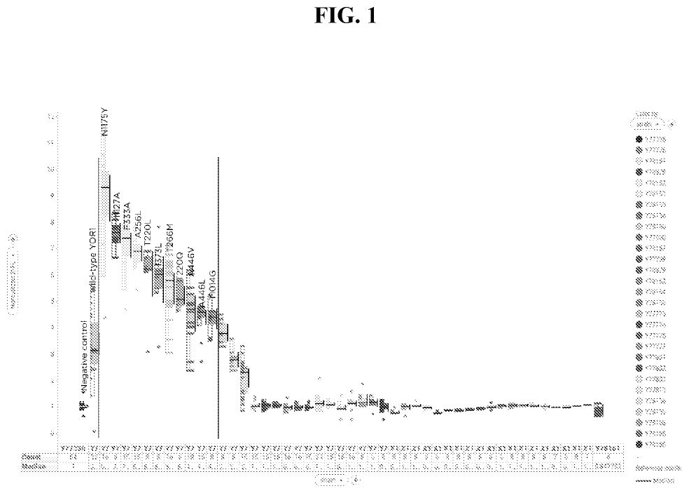

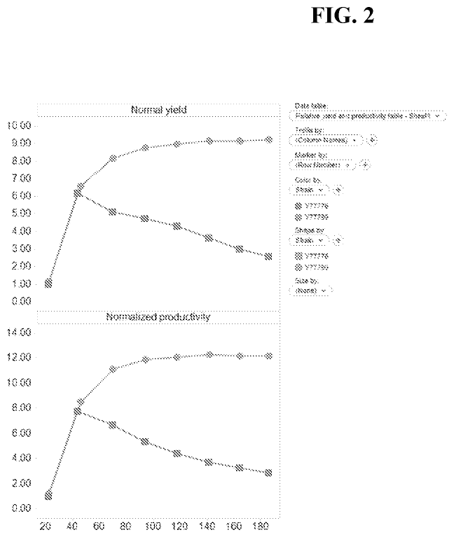

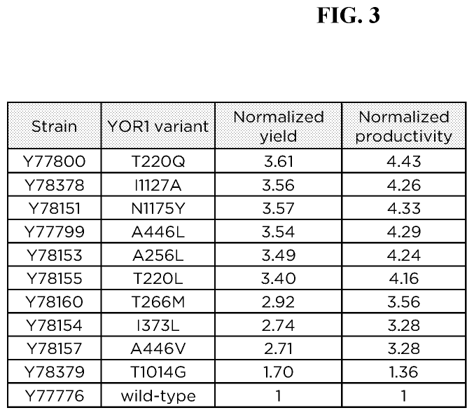

The initial screen of 5952 individual isolates yielded 270 hits (see FIG. 1): 171 hits showing increased 2′-FL titer and increased 2′-FL/DFL ratio; 67 hits showing increased 2′-FL titer and lowest lactose titer remaining; and 32 hits showing highest increase in total FL equivalents. The 270 hits were further screened and of those 20 were promoted to the next level (see FIG. 2). Of these 20 a subset of 11 variants that showed significant improvement for 2′-FL titers compared to the parental YOR1 were selected for further analysis ((TABLE 1) and FIG. 3).

TABLE 1
Eleven strains with unique YOR1 variants that showed
significant improvement for 2′-FL titers.

| Strain | Description |
|---|---|
| Y78151 | Y77335 with YOR1_N1175Y |
| Y78378 | Y77335 with YOR1_I1127A |
| Y78152 | Y77335 with YOR1_F333A |
| Y78153 | Y77335 with YOR1_A256L |
| Y78155 | Y77335 with YOR1_T220L |
| Y78154 | Y77335 with YOR1_I373L |
| Y78160 | Y77335 with YOR1_T266M |
| Y77800 | Y77335 with YOR1_T220Q |
| Y78157 | Y77335 with YOR1_A446V |
| Y77799 | Y77335 with YOR1_A446L |
| Y78379 | Y77335 with YOR1_T1014G |

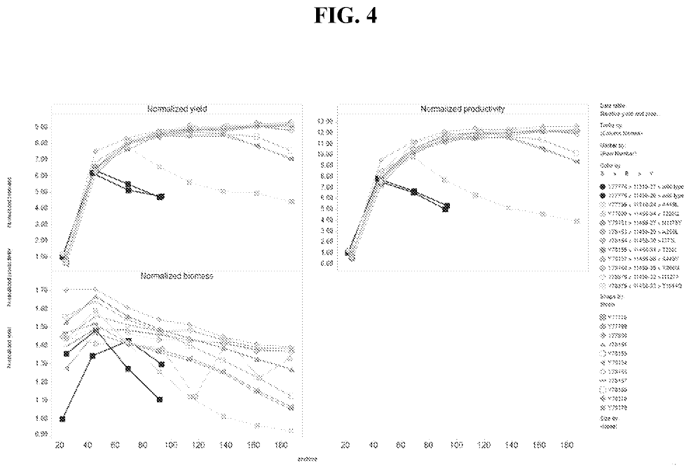

A strain expressing YOR1_A446L (Y77799) was compared to a strain expressing wild-type YOR1 (Y77776) in a two-liter fermentation tank (See FIG. 4). Carbon flux, 2′-FL productivity, 2′-FL yield, and residual lactose were measured over time. As shown in FIG. 4, the strain expressing the variant YOR1_A446L had improved flux, productivity, yield, and lactose utilization compared to the strain expressing parental YOR1.

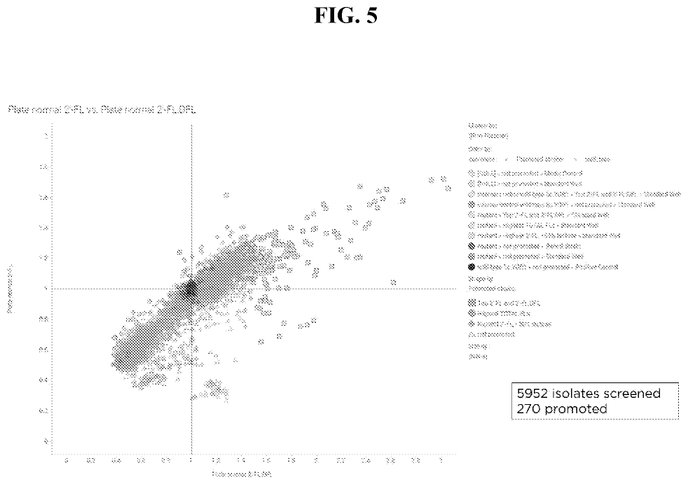

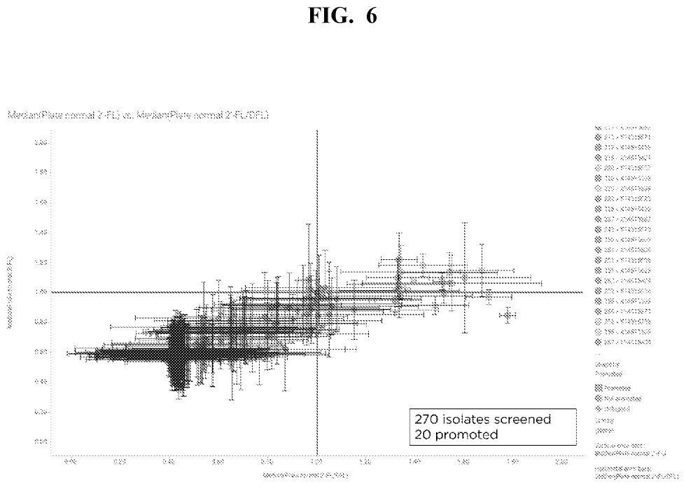

Ten strains each carrying a different YOR1 variant (T220Q, I1127A, N1175Y, A446L, A256L, T220L, T266M, I373L, A446V, and T1014G) were compared to strains expressing wild-type YOR1 in two-liter fermentation tanks. As shown in FIG. 5, the ten variants had improved 2′-FL yield and productivity compared to strains expressing wild-type YOR1. FIG. 6 shows the performance of the tested strains over time. In this set of experiments 2′-FL producing strains that express YOR1_I1127A or YOR1_T220Q were the top two performing strains with respect to 2′-FL performance.

### Example 2. Directed Evolution of YOR1 Using Error-Prone PCR and Growth Selection

A directed evolution-based approach was carried out using error-prone PCR of the 2′-FL transporter, YOR1. First, a growth competition-based assay was performed to evaluate if transport of 2′-FL would confer a specific adaptive advantage to a strain containing transporter when compared to its counterpart with no transporter. Cultures of strains with or without transporters were mixed equally in production conditions and plated at either 24, 48 or 72 h to get single colonies. The individual colonies were then subjected to CPCR to verify genotypes. After 72 h of growth in production conditions, the strain with YOR1 outcompeted its counterpart without transporter. This growth-based assay was used to enrich for mutants of YOR1 that had a selective growth advantage by exporting more 2′-FL out of the cell. For error-prone PCR, the DNA coding sequence of YOR1 was subjected to mutagenesis using GeneMorph II Random Mutagenesis Kit (Agilent Technologies, Inc). The resulting library was transformed into a 2′-FL producing strain alongside wild type version of YOR1 as a control. After the overnight recovery, the cultures were transferred to production medium supplemented with antibiotic for selective growth of transformed population. The OD₆₀₀ of the culture was monitored regularly and the culture was propagated serially with fresh medium containing antibiotic making sure that the cells never reached carbon starvation. The culture was plated every 24 hours on agar medium containing antibiotic to obtain single colonies. Approximately 90 colonies from each timepoint were tested for 2′-FL and DFL production. 2′-FL/DFL ratio was used to screen for increased export activity as previously mentioned in Example 1. This was based on the rationale that if 2′-FL was quickly exported out of the cytoplasm, the fucosyltransferase would not be able to fucosyllate it further to make DFL resulting in increased amount of 2′-FL relative to the DFL. Several colonies showed improved transport of 2′-FL as measured by increase in 2′-FL/DFL ratio. A couple of isolates also showed improved 2′-FL/DFL ratio over wild type enzyme. Overall, population from 120 h and 140 h timepoints showed an improvement in transport activity. Isolates with highest improvement from 120 h were sequenced using whole genome sequencing to reveal the causal mutations. All the isolates had 3 SNPs in the YOR1 coding sequence which led to change in the amino acid sequence. These were identified as Q64H, L795Q and L1167S.

Illustrative Sequences

SEQ ID NO: 1 (Saccharomyces cerevisiae YOR1)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 2 (YOR1_A446L)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSILSLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 3 (YOR1_T220Q)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCQSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 4 (YOR1_I1127A)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAAAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 5 (YOR1_N1175Y)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTEYDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 6 (YOR1_A256L)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGLCLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 7 (YOR1_T220L)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCLSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 8 (YOR1_T266M)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLMFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 9 (YOR1_1373L)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLLLGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 10 (YOR1_A446V)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIVSLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 11 (YOR1_T1014G)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMGSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 12 (YOR1_Q64H)
MTITVGDAVSETELENKSQNVVLSPKASASSDISTDVDKDTSSSWDDKSL
LPTGEYIVDRNKPHTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 13 (YOR1_L795Q)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDEQKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 14 (YOR1_L1167S)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGFPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTISRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAIIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS
SEQ ID NO: 15 (YOR1_F333A)
MTITVGDAVSETELENKSQNVVLSPKASASSDISTDVDKDTSSSWDDKSL
LPTGEYIVDRNKPQTYLNSDDIEKVTESDIFPQKRLFSFLHSKKIPEVPQ
TDDERKIYPLFHTNIISNMFFWWVLPILRVGYKRTIQPNDLFKMDPRMSI
ETLYDDFEKNMIYYFEKTRKKYRKRHPEATEEEVMENAKLPKHTVLRALL
FTFKKQYFMSIVFAILANCTSGFNPMITKRLIEFVEEKAIFHSMHVNKGI
GYAIGACLMMFVNGLTFNHFFHTSQLTGVQAKSILTKAAMKKMFNASNYA
RHCFPNGKVTSFVTTDLARIEFALSFQPFLAGAPAILAICIVLLIVNLGP
IALVGIGIFFGGFFISLFAFKLILGFRIAANIFTDARVTMMREVLNNIKM
IKYYTWEDAYEKNIQDIRTKEISKVRKMQLSRNFLIAMAMSLPSIASLVT
FLAMYKVNKGGRQPGNIFASLSLFQVLSLQMFFLPIAIGTGIDMIIGLGR
LQSLLEAPEDDPNQMIEMKPSPGFDPKLALKMTHCSFEWEDYELNDAIEE
AKGEAKDEGKKNKKKRKDTWGKPSASTNKAKRLDNMLKDRDGPEDLEKTS
FRGFKDLNFDIKKGEFIMITGPIGTGKSSLLNAMAGSMRKTDGKVEVNGD
LLMCGYPWIQNASVRDNIIFGSPFNKEKYDEVVRVCSLKADLDILPAGDM
TEIGERGITLSGGQKARINLARSVYKKKDIYLFDDVLSAVDSRVGKHIMD
ECLTGMLANKTRILATHQLSLIERASRVIVLGTDGQVDIGTVDELKARNQ
TLINLLQFSSQNSEKEDEEQEAVVAGELGQLKYESEVKELTELKKKATEM
SQTANSGKIVADGHTSSKEERAVNSISLKIYREYIKAAVGKWGFIALPLY
AILVVGTTFCSLFSSVWLSYWTENKFKNRPPSFYMGLYSFFVFAAFIFMN
GQFTILCAMGIMASKWLNLRAVKRILHTPMSYIDTTPLGRILNRFTKDTD
SLDNELTESLRLMTSQFANIVGVCVMCIVYLPWFAIAIPFLLVIFVLIAD
HYQSSGREIKRLEAVQRSFVYNNLNEVLGGMDTIKAYRSQERFLAKSDFL
INKMNEAGYLVVVLQRWVGIFLDMVAIAFALIITLLCVTRAFPISAASVG
VLLTYVLQLPGLLNTILRAMTQTENDMNSAERLVTYATELPLEASYRKPE
MTPPESWPSMGEIIFENVDFAYRPGLPIVLKNLNLNIKSGEKIGICGRTG
AGKSTIMSALYRLNELTAGKILIDNVDISQLGLFDLRRKLAHIPQDPVLF
RGTIRKNLDPFNERTDDELWDALVRGGAIAKDDLPEVKLQKPDENGTHGK
MHKFHLDQAVEEEGSNFSLGERQLLALTRALVRQSKILILDEATSSVDYE
TDGKIQTRIVEEFGDCTILCIAHRLKTIVNYDRILVLEKGEVAEFDTPWT
LFSQEDSIFRSMCSRSGIVENDFENRS

What is claimed is:

1. A yeast cell genetically modified to produce one or more human milk oligosaccharides, wherein the yeast cell comprises (i) a heterologous nucleic acid encoding a variant human milk oligosaccharide (HMO) ABC transporter polypeptide having one or more amino acid substitutions compared to SEQ ID NO: 1; and (ii) one or more heterologous nucleic acids that each independently encode at least one enzyme of a human milk oligosaccharide biosynthetic pathway.

2. The yeast cell of claim 1, wherein the amino acid substitution is selected from A446L, T220Q, I1127A, N1175Y, A256L, T220L, T226M, I373L, A446V, T1014G, Q64H, L795Q, L1167S, and F333A.

3. The yeast cell of claim 1, wherein the variant ABC transporter polypeptide comprises an amino acid sequence selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

4. The yeast cell of claim 3, wherein the variant ABC transporter polypeptide comprises an amino acid sequence of SEQ ID NO: 2.

5. The yeast cell of claim 3, wherein the variant ABC transporter polypeptide comprises an amino acid sequence of SEQ ID NO: 3.

6. The yeast cell of claim 3, wherein the variant ABC transporter polypeptide comprises an amino acid sequence of SEQ ID NO: 4.

7. The yeast cell of claim 3, wherein the variant ABC transporter polypeptide comprises an amino acid sequence of SEQ ID NO: 5.

8. The yeast cell of claim 3, wherein the variant ABC transporter polypeptide comprises an amino acid sequence of SEQ ID NO: 6.

9. The yeast cell of claim 3, wherein the variant ABC transporter polypeptide comprises an amino acid sequence of SEQ ID NO: 7.

10. The yeast cell of claim 3, wherein the variant ABC transporter polypeptide comprises an amino acid sequence of SEQ ID NO: 8.

11. The yeast cell of claim 3, wherein the variant ABC transporter polypeptide comprises an amino acid sequence of SEQ ID NO: 9.

12. The yeast cell of claim 3, wherein the variant ABC transporter polypeptide comprises an amino acid sequence of SEQ ID NO: 10.

13. The yeast cell of claim 3, wherein the variant ABC transporter polypeptide comprises an amino acid sequence of SEQ ID NO: 11.

14. The yeast cell of claim 3, wherein the variant ABC transporter polypeptide comprises an amino acid sequence of SEQ ID NO: 12.

15. The yeast cell of claim 3, wherein the variant ABC transporter polypeptide comprises an amino acid sequence of SEQ ID NO: 13.

16. The yeast cell of claim 3, wherein the variant ABC transporter polypeptide comprises an amino acid sequence of SEQ ID NO: 14.

17. The yeast cell of claim 3, wherein the variant ABC transporter polypeptide comprises an amino acid sequence of SEQ ID NO: 15.

18. The yeast cell of claim 1, wherein the host cell produces the HMO at a yield at least 1.1 times greater than the yield of a host cell lacking the ABC transporter polypeptide.

19. The yeast cell of claim 1, wherein the host cell produces the HMO at a productivity at least 1.1 times greater than the productivity of a host cell lacking the ABC transporter polypeptide.

20. The yeast cell of any one of the preceding claims, wherein the ABC transporter exports the human milk oligosaccharide 2′-fucosyllactose.

21. The yeast cell of any one of the preceding claims, wherein the one or more human milk oligosaccharides comprise 2′-fucosyllactose.

22. The yeast cell of any one of the preceding claims, wherein the heterologous nucleic acid encoding the ABC transporter polypeptide is integrated into the genome of the yeast cell and/or the one or more heterologous nucleic acids that each independently encode at least one enzyme of a human milk oligosaccharide biosynthetic pathway is integrated into the genome of the yeast cell.

23. The yeast cell of any one of the preceding claims, wherein the yeast cell is a Saccharomyces sp. or a Kluveromyces sp.

24. The yeast cell of claim 23, wherein the yeast cell is a Saccharomyces cerevisiae cell.

25. A method of producing one or more human milk oligosaccharides, the method comprising culturing a population of genetically modified yeast cells of any one of claims 1-24 in a culture medium under conditions suitable for the yeast cells to produce the one or more human milk oligosaccharides.

26. The method of claim 25, wherein the one or more human milk oligosaccharides comprise 2′-fucosyllactose.

27. A fermentation composition comprising:
a population of genetically modified yeast cells comprising the yeast cell of any one of claims 1-24; and a culture medium comprising one or more human milk oligosaccharides produced from the yeast cells.

28. A method of recovering one or more human milk oligosaccharides from the fermentation composition of claim 27, the method comprising:
separating at least a portion of the population of genetically modified yeast cells from the culture medium; and contacting the separated yeast cells with a heated aqueous wash liquid; and
removing the wash liquid from the separated yeast cells.


SEQUENCE LISTING (15 sequences)

SEQ ID NO: 1 - protein, 1477 aa, Saccharomyces cerevisiae
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 2 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSILSLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 3 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCQSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 4 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAAAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 5 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTEYDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 6 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGLCLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 7 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCLSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 8 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLMFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 9 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLLLGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 10 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIVSLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 11 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMGSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 12 - protein, 1477 aa, synthetic construct
MTITVGDAVSETELENKSQNVVLSPKASASSDISTDVDKDTSSSWDDKSLLPTGEYIVDRNKPHTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 13 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDEQKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 14 - protein, 1477 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTISRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 15 - protein, 1478 aa, synthetic construct
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGAFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS